(19) United States
(12) Patent Application Publication
(10) Pub. No.: US 2026/0345224 A1
(43) Pub. Date: Nov. 5, 2026

(54) Electrosurgical Electrode and Electrosurgical Devices Having Adjustable Configurations

(71) Applicant: Stryker European Operations Limited, Cork (IE)

(72) Inventors: Micheal Burke, Cork (IE); Paul Sheridan, Wexford (IE); Laura Constance Frey, Belfast (IE); Stephen Faul, Cork (IE)

(21) Appl. No.: 19/119,968

(22) PCT Filed: Sep. 29, 2023
(86) PCT No.: PCT/IB2023/000597

§ 371 Date: Apr. 10, 2025

Related U.S. Application Data
(60) Provisional application No. 63/414,822, filed on Oct. 10, 2022.
(60) Provisional application No. 63/438,153, filed on Jan. 10, 2023.

(51) Int. Cl. A61B 18/14; A61B 18/00; A61B 18/12

(52) U.S. Cl. CPC A61B 18/14; A61B 18/1206; A61B 2018/00077; A61B 2018/00083; A61B 2018/00178; A61B 2018/00202; A61B 2018/00922; A61B 2018/1412; A61B 2018/1417; A61B 2218/008

(57) ABSTRACT

An example electrosurgical electrode includes a first end effector at a first end, and a second end effector at a second end. The first end is opposite the second end. The electrosurgical electrode has a longitudinal axis extending between the first end and the second end. A mount is between the first end effector and the second end effector. The mount is configured to couple the electrosurgical electrode to a housing of an electrosurgical device.

### CROSS-REFERENCE TO RELATED APPLICATIONS

The present application claims the benefit of priority of U.S. Provisional Application No. 63/438,153, filed Jan. 10, 2023, and U.S. Provisional Application No. 63/414,822, filed Oct. 10, 2022, the contents of which are hereby incorporated by reference in their entirety.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of an electrosurgical system, according to an example.

FIG. 2 depicts a simplified block diagram of an electrosurgical electrode, according to an example.

FIG. 3 depicts a perspective view of the electrosurgical electrode shown in FIG. 2, according to an example.

FIG. 4A depicts an implementation of the electrosurgical device shown in FIG. 1 with the electrosurgical electrode shown in FIG. 3 in a first state, according to an example.

FIG. 4B depicts an assembly of components for the electrosurgical device shown in FIG. 4A, according to an example.

FIG. 4C depicts an implementation of the electrosurgical device shown in FIG. 1 with the electrosurgical electrode shown in FIG. 3 in a second state, according to an example.

FIG. 4D depicts an assembly of components for the electrosurgical device shown in FIG. 4C, according to an example.

FIG. 5 depicts a simplified block diagram of an electrosurgical system, according to another example.

FIG. 6A depicts an implementation of the electrosurgical device shown in FIG. 5 with the electrosurgical electrode shown in FIG. 3 in a first state, according to an example.

FIG. 6B depicts an assembly of components for the electrosurgical device shown in FIG. 6A, according to an example.

FIG. 6C depicts an implementation of the electrosurgical device shown in FIG. 5 with the electrosurgical electrode shown in FIG. 3 in a second state, according to an example.

FIG. 6D depicts an assembly of components for the electrosurgical device shown in FIG. 4C, according to an example.

FIG. 7A depicts an implementation of the electrosurgical device shown in FIG. 5, according to another example.

FIG. 7B depicts an assembly of components for the electrosurgical device shown in FIG. 7A, according to an example.

FIG. 8A depicts an implementation of the electrosurgical device shown in FIG. 5, according to another example.

FIG. 8B depicts an assembly of components for the electrosurgical device shown in FIG. 8A, according to an example.

FIG. 9A depicts an electrosurgical electrode that can be used with the electrosurgical device shown in FIG. 1 and/or FIG. 5, according to another example.

FIG. 9B depicts an electrosurgical electrode that can be used with the electrosurgical device shown in FIG. 1 and/or FIG. 5, according to another example.

FIG. 9C depicts an electrosurgical electrode that can be used with the electrosurgical device shown in FIG. 1 and/or FIG. 5, according to another example.

FIG. 9D depicts an electrosurgical electrode that can be used with the electrosurgical device shown in FIG. 1 and/or FIG. 5, according to another example.

FIG. 9E depicts an electrosurgical electrode that can be used with the electrosurgical device shown in FIG. 1 and/or FIG. 5, according to another example.

FIG. 10 depicts a flowchart for a method of operating an electrosurgical device, according to an example.

FIG. 11 depicts a flowchart for a method of operating an electrosurgical device that can be performed with the process shown in FIG. 10, according to an example.

FIG. 12 depicts a flowchart for a method of operating an electrosurgical device that can be performed with the process shown in at least FIG. 10, according to an example.

FIG. 13 depicts a flowchart for a method of operating an electrosurgical device that can be performed with the process shown in at least FIG. 10, according to an example.

FIG. 14 depicts a flowchart for a method of operating an electrosurgical device that can be performed with the process shown in at least FIG. 10, according to an example.

FIG. 15 depicts a flowchart for a method of operating an electrosurgical device that can be performed with the process shown in at least FIG. 10, according to an example.

FIG. 16 depicts a flowchart for a method of operating an electrosurgical device that can be performed with the process shown in at least FIG. 10, according to an example.

FIG. 17 depicts a flowchart for a method of operating an electrosurgical device that can be performed with the process shown in at least FIG. 10, according to an example.

FIG. 18 depicts a flowchart for a method of forming an electrosurgical device, according to an example.

FIG. 19 depicts a flowchart for a method of operating an electrosurgical device, according to an example.

FIG. 20 depicts a flowchart for a method of operating an electrosurgical device that can be performed with the process shown in at least FIG. 19, according to an example.

FIG. 21 depicts a flowchart for a method of operating an electrosurgical device that can be performed with the process shown in at least FIG. 19, according to an example.

FIG. 22 depicts a flowchart for a method of forming an electrosurgical electrode, according to an example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

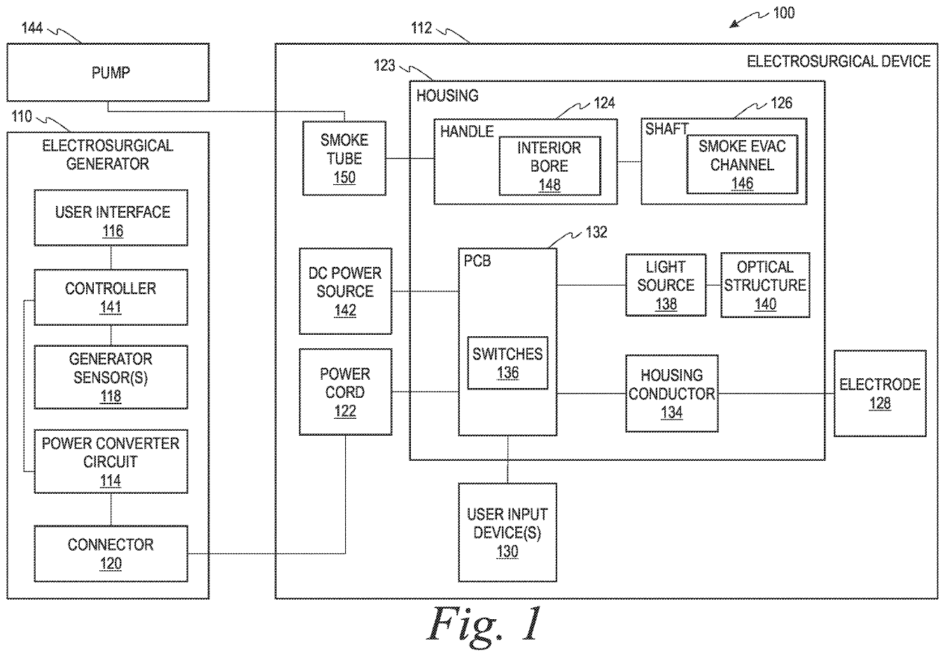

Referring to FIG. 1, an electrosurgical system 100 is shown according to an example. As shown in FIG. 1, the electrosurgical system 100 includes an electrosurgical generator 110 and an electrosurgical device 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more generator sensors 118 that can sense one or more conditions related to the electrosurgical energy and/or the target tissue. As examples, the generator sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the generator sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical device 112. For example, the electrosurgical device 112 can include a power cord 122 having a plug, which can be coupled to a socket of the connector 120 of the electrosurgical generator 110. In this arrangement, the electrosurgical generator 110 can supply the electrosurgical energy to the electrosurgical device 112 via the coupling between the connector 120 of the electrosurgical generator 110 and the power cord 122 of the electrosurgical device 112.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical device 112 can include a housing 123 having a proximal end and a distal end, and an electrosurgical electrode 128 extending from the distal end of the housing 123. The housing 123 can be an elongated structure in and/or on which components of the electrosurgical device 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore, and a shaft 126 extending in a distal direction from the handle 124. In general, the handle 124 can be configured to facilitate a user gripping and manipulating the electrosurgical device 112 while performing electrosurgery. For example, the handle 124 can have a shape and/or a size that can facilitate a user performing electrosurgery by manipulating the electrosurgical device 112 using a single hand. In one implementation, the handle 124 can have a shape and/or a size that facilitates the user holding the electrosurgical device 112 in a writing utensil gripping manner (e.g., the electrosurgical device 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical device 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical device 112). In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical device 112, which can facilitate performing electrosurgery at a plurality of different depths within tissue (e.g., due to different anatomical shapes and/or sizes of patients) and/or at a plurality of different angles. In other examples, the electrosurgical electrode 128 can be fixedly coupled to the handle 124 such that the shaft 126 is axially movable relative to both the electrosurgical electrode 128 and the handle 124.

In some implementations, the electrosurgical electrode 128 can additionally or alternatively be rotatable about an axis of rotation that is parallel to the longitudinal axis of the electrosurgical device 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124. Rotating the electrosurgical electrode 128 relative to the handle 124 can facilitate adjusting an angle of the electrosurgical electrode 128 relative to one or more user input device(s) 130 of the electrosurgical device 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees, rotatable by 270 degrees, or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts).

As shown in FIG. 1, the electrosurgical device 112 can include one or more user input devices 130 that are operable to control operation of the electrosurgical device 112 and/or the electrosurgical generator 110. For instance, the user input device(s) 130 can be operable to select between the modes of operation of the electrosurgical device 112 and/or the electrosurgical generator 110. In one implementation, the user input device(s) 130 can be configured to select between a cutting mode of operation and a coagulation mode of operation. Responsive to actuation of the user input device(s) 130 of the electrosurgical device 112, the electrosurgical device 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: a printed circuit board 132 (e.g., a flexible printed circuit board) and/or one or more housing conductors 134 that are configured to conduct electrosurgical energy from the power cord 122 to the electrosurgical electrode 128. One or more of the electrical components can be positioned in the inner bore defined by the handle 124 and/or in the inner cavity defined by the shaft 126.

Within examples, the user input device(s) 130 can include one or more buttons on an exterior surface of the handle 124. Each button of the user input device(s) 130 can be operable to actuate a respective one of a plurality of switches 136 of the printed circuit board 132. In general, the switches 136 and/or the printed circuit board 132 are operable to control a supply of the electrosurgical energy from the electrosurgical generator 110 to the electrosurgical electrode 128. For instance, in one implementation, when each button is operated (e.g., depressed), the respective switch 136 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 136 associated with the button can close the switch 136 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switch(es) 136 to (ii) the electrosurgical electrode 128 by the housing conductor(s) 134. As such, as shown in FIG. 1, the printed circuit board 132 can be coupled to the power cord 122, the printed circuit board 132 can be coupled to the housing conductor(s) 134, and the housing conductor(s) 134 can be coupled to the electrosurgical electrode 128. In this arrangement, the housing conductor(s) 134 can conduct the electrosurgical energy to the electrosurgical electrode 128. The switch(es) 136 can be coupled to the printed circuit board 132 in some examples.

In general, the housing conductor(s) 134 can each include one or more electrically conductive elements that provide an electrically conductive bus for supplying the electrosurgical energy to the electrosurgical electrode 128. In some examples, the electrical components of the electrosurgical device 112 can be electrically coupled to each other in a manner that is suitable to supply electrosurgical energy from the power cord 122 to the electrosurgical electrode 128 while (i) the shaft 126 and/or the electrosurgical electrode 128 telescopically moves relative to the handle 124, and/or (ii) the electrosurgical electrode 128 rotates relative to the handle 124.

Although the electrosurgical device 112 includes the user input device(s) 130 in FIG. 1, the user input device(s) 130 can be separate from the electrosurgical device 112 in another example. For instance, the user input device(s) 130 can additionally or alternatively include one or more foot pedals that are actuatable to control operation of the electrosurgical device 112 as described above. The foot pedal(s) can be communicatively coupled to the electrosurgical generator 110 to provide a signal responsive to actuation of the foot pedal(s).

As shown in FIG. 1, in some implementations, the electrosurgical device 112 can additionally include one or more light sources 138 that are configured to emit light. In some examples that include the light source(s) 138, the user input device 130 can be operable to cause the light source(s) 138 to generate light that can be emitted by the electrosurgical device 112 to illuminate an area of interest (e.g., a target tissue at the surgical site). In some implementations, the light source(s) 138 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 138 can be optically coupled to an optical structure 140, which is configured to receive the light emitted by the light source(s) 138 and transmit the light in a distal direction toward a surgical site to illuminate the surgical site while performing electrosurgery using the electrosurgical electrode 128. Although arranging the light source(s) 138 to directly illuminate a surgical field can help, for instance, to reduce a cost of manufacture, transmitting the light using the optical structure 140 can help to improve a quality of light transmitted from the electrosurgical device 112 (e.g., by providing light with improved uniformity and/or reduced heat generation).

As examples, in implementations that include the optical structure 140, the optical structure 140 can include at least one optical structure selected from among a group consisting of an optical lens, a non-fiber optic optical waveguide, and an optical fiber. When the optical structure 140 includes the optical lens (e.g., a parabolic reflector lens, an aspheric lens, and/or a Fresnel lens), the optical structure 140 can help to direct the light emitted by the light source 138 in the distal direction and thereby improve a quality of the light illuminating the surgical site. The optical structure 140 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 140 can additionally or alternatively include other light shaping optical elements such as, for instance, a plurality of facets, one or more prisms, and/or one or more optical gratings. Although the optical structure 140 can help to improve a quality of the light directed to the surgical site, the electrosurgical device 112 can omit the optical structure 140 and instead emit the light from the light source 138 directly to the surgical field without transmitting the light through the optical structure 140 in other examples.

In FIG. 1, the light source 138 can be coupled to the shaft 126. As such, the light source 138 can also move telescopically with the shaft 126 relative to the handle 124. However, in other examples, the light source 138 can be in the interior bore of the handle 124 and/or coupled to an exterior surface of the handle 124. As examples, the light source 138 can include one or more light emitting diodes (LEDs), organic light emitting diodes (OLEDs), optical fibers, non-fiber optic waveguides, and/or lenses. Additionally, for example, the light source 138 can include a LED printed circuit board having one or more light sources (e.g., LEDs).

The optical structure 140 can be at a distal end of the shaft 126. In some examples, the optical structure 140 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical device 112 relative to the target tissue. However, in other examples, the optical structure 140 can extend partially but not fully around the electrosurgical electrode 128.

In implementations that include the light source 138, the user input device(s) 130, the printed circuit board 132, the switches 136, and/or the housing conductor(s) 134 can additionally supply an electrical power from a direct current (DC) power source 142 to the light source 138. In one example, the DC power source 142 can include a battery disposed in the handle 124, the plug of the power cord 122, and/or a battery receptacle located along the power cord 122 between the handle 124 and the plug. Although the electrosurgical device 112 includes the DC power source 142 in FIG. 1, the DC power source 142 can be separate and distinct from the electrosurgical device 112 in other examples. For instance, in another example, the electrosurgical generator 110 can include the DC power source 142.

Additionally, in implementations that include the light source 138, the user input device(s) 130 can be operable to cause the light source 138 to emit the light. In one example, the user input device(s) 130 can include a button that independently controls the light source 138 separate from the button(s) that control the electrosurgical operational modes of the electrosurgical device 112. In another example, the user input device(s) 130 and the printed circuit board 132 can be configured such that operation of the button(s) that control the electrosurgical operational mode simultaneously control operation of the light source 138 (e.g., the light source 138 can be automatically actuated to emit light when a button is operated to apply the electrosurgical energy at the electrosurgical electrode 128).

As shown in FIG. 1, responsive to operation of the user input device(s) 130 to actuate the light source 138, the DC power source 142 can supply the electrical power (e.g., a DC voltage) to the light source 138 via the printed circuit board 132 and/or the housing conductor(s) 134. In this implementation, one or more of the conductive elements of the housing conductor(s) 134 can be configured to supply the electrical power from the DC power source 142 to the light source 138 and/or return the electrical power from the light source 138 to the DC power source 142. Accordingly, the housing conductor(s) 134 can additionally or alternatively assist in providing electrical communication between the DC power source 142 and the light source 138 as the shaft 126 and the light source 138 telescopically move relative to the handle 124.

Although the user input device(s) 130 on the handle 124 can be operated to control the operation of the light source 138 in the examples described above, the light source 138 can be additionally or alternatively operated by one or more user input device(s) on the electrosurgical generator 110 (e.g., via the user interface 116) and/or on the plug of the power cord 122.

In some examples, the electrosurgical device 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 144 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 144 and the electrosurgical generator 110 can be provided as separate devices or integrated in a single device (e.g., in a common housing).

As shown in FIG. 1, the shaft 126 can include a smoke evacuation channel 146 in the inner cavity of the shaft 126. The smoke evacuation channel 146 can also include one or more smoke inlets at one or more positions around the electrosurgical electrode 128. In some examples, the smoke evacuation channel 146 can include a plurality of smoke inlets on opposing sides of the electrosurgical electrode 128. In this arrangement, the smoke inlet of the smoke evacuation channel can help to receive surgical smoke into the smoke evacuation channel 146 in a plurality of rotational alignments of the electrosurgical electrode 128 relative to the handle 124 and/or the electrosurgical device 112 relative to the target tissue.

In an example, the smoke evacuation channel 146 of the shaft 126 defines a first portion of a smoke flow path, and an interior bore 148 of the handle 124 defines a second portion of a smoke flow path. In this arrangement, the surgical smoke can be received from the surgical site into the smoke evacuation channel 146 of the shaft 126, and flow proximally along the smoke evacuation channel 146 to the interior bore 148 of the handle 124. In the interior bore 148 of the handle 124, the smoke can further flow to a smoke tube 150 that is coupled to a proximal end of the handle 124 and configured to convey smoke from the handle 124 to the suction pump 144.

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

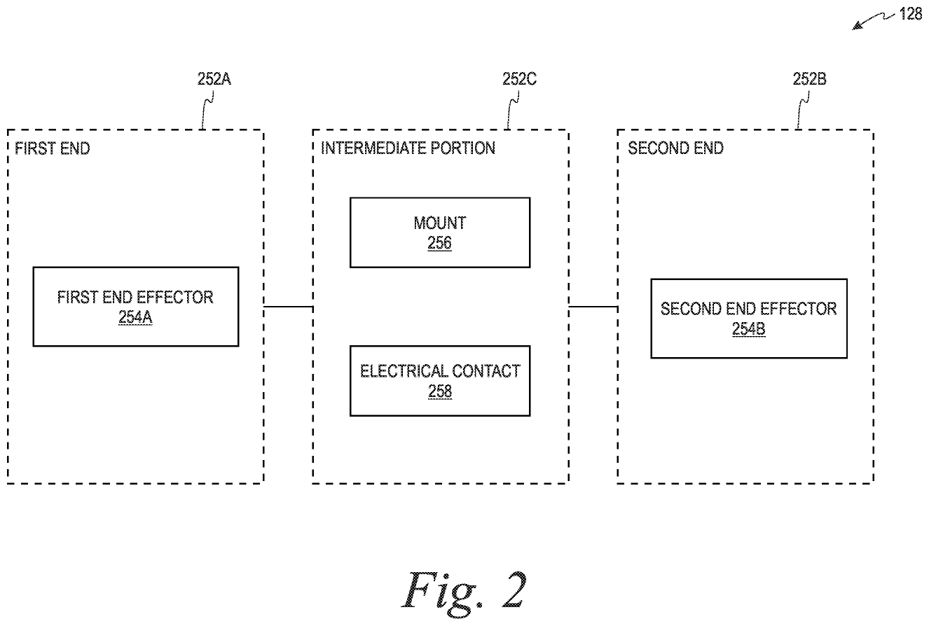

Referring now to FIG. 2, a simplified block diagram of the electrosurgical electrode 128 is shown according to an example. The electrosurgical electrode 128 has a longitudinal axis extending between a first end 252A of the electrosurgical electrode 128 and a second end 252B of the electrosurgical electrode 128. As such, the electrosurgical electrode 128 can be elongated in an axial direction between the first end 252A of the electrosurgical electrode 128 and the second end 252B of the electrosurgical electrode 128.

As shown in FIG. 2, the electrosurgical electrode 128 can have a first end effector 254A at the first end 252A, and a second end effector 254B at the second end 252B. The electrosurgical electrode 128 can also include a mount 256 between the first end effector 254A and the second end effector 254B. For instance, the mount 256 can be at an intermediate portion 252C of the electrosurgical electrode 128, which is between the first end 252A and the second end 252B. The mount 256 is configured to releasably and reversibly couple the electrosurgical electrode 128 to the housing 123 of the electrosurgical device 112. For instance, the electrosurgical electrode 128 can be configured to releasably couple to the housing 123 in (i) a first state in which the first end effector 254A is in an interior cavity of the housing 123 and the second end effector 254B extends from the distal end of the housing 123, and (ii) a second state in which the second end effector 254B is in the interior cavity of the housing 123 and the first end effector 254A extends from the distal end of the housing 123. In this arrangement, when the electrosurgical electrode 128 is coupled to the housing 123, one of the first end effector 254A and the second end effector 254B extends distally from the distal end of the housing 123 to apply electrosurgical energy to a target tissue during an electrosurgical operation, and the other of the first end effector 254A and the second end effector 254B is housed within the interior cavity of the housing 123 can is not used during the electrosurgical operation.

In some examples, the first end effector 254A and the second end effector 254B can have the same configuration. For instance, the first end effector has a first configuration, the second end effector has a second configuration, and the first configuration and the second configuration are the same as each other in a size, a shape, and a material. In other words, in these examples, the first end effector 254A and the second end effector 254B can be identical to each other. In this example, the electrosurgical electrode 128 can be used in the first state initially to use the first end effector 254A, and the electrosurgical electrode 128 can then be switched from the first state in the housing 123 to the second state to use the second end effector 254B. The switch may be performed after some degradation in the performance of the first end effector 254A (e.g., due to char build-up). This can help to improve operation efficiency as compared to other electrosurgical devices, which require swapping out the entire electrosurgical electrode with a new one (e.g., including obtaining an entirely different electrosurgical electrode from a package and disposing of the previous electrosurgical electrode in a sharps bin).

In other examples, the first end effector 254A and the second end effector 254B can have different configurations. In such examples, the first end effector 254A can have a first configuration, the second end effector 254B has a second configuration, and the first configuration and the second configuration can be different from each other in at least one property selected from a group of properties consisting of: a size, a shape, and a material. As such, a practioner can selectively couple the electrosurgical electrode 128 to the housing 123 in the first state or the second state to select the configuration that will achieve a desired surgical effect, and/or the practioner can selectively switch between the first state and the second state to modify the surgical effect provided by the electrosurgical device 112 to a target tissue during an electrosurgical operation.

In one implementation, the first end effector 254A and the second end effector 254B can be different types of electrodes selected from among a group consisting of: a blade type electrode, a ball tip type electrode, and a needle tip type electrode. A blade type electrode can be used for cutting, coagulating, ablating, and/or sealing the tissue. A ball type electrode may be beneficial for coagulation and fulguration operations. A needle tip type electrode may be beneficial for delivering precise, concentrated electrosurgical energy during an incision, excision, and/or pinpoint coagulation operation.

In another example implementation, the first end effector 254A and the second end effector 254B can be the same type electrode, but have different sizes. For instance, the first end effector 254A can have a first length, the second end effector 254B can have a second length, and the first length can be different than the second length. This can facilitate performing electrosurgery at a plurality of different depths within tissue (e.g., due to different anatomical shapes and/or sizes of patients) and/or at a plurality of different angles.

As another example implementation, a tip of the first end effector 254A can be a ball of a first diameter, a tip of the second end effector 254B can be a ball of a second diameter, and the first diameter can be different than the second diameter. In another implementation, the first end effector 254A can be a needle of a first gauge, the second end effector 254B can be a needle of a second gauge, and the first gauge can be different than the second gauge.

In another implementation, the first end effector 254A can be a first electrosurgical blade having first dimensions, the second end effector 254B can be a second electrosurgical blade having second dimensions, and the first dimensions can be different than the second dimensions. Each electrosurgical blade can include (i) a first lateral side (e.g., a surface or an edge), (ii) a second lateral surface (e.g., a surface or an edge) opposite the first lateral surface, (iii) a first major surface extending between the first lateral side and the second lateral side on a first side of the electrosurgical blade, and (iv) a second major surface extending between the first lateral side and the second lateral side on a second side of the electrosurgical blade that is opposite the first side. The first lateral side and the second lateral side have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the electrosurgical blade) and a width (e.g., a dimension between the first latera side and the second lateral side). In this implementation, the first end effector 254A and the second end effector 254B can have different dimensions in at least one dimension selected from a group consisting of: the thickness, the length, and the width.

As described above, in some examples, the first end effector 254A and the second end effector 254B can have different or the same materials. For instance, in some examples, the first end effector 254A and/or the second end effector 254B of the electrosurgical electrode 128 can also include an outer layer of material covering the electrosurgical substrate at the first end effector 254A and/or the second end effector 254B. The outer layer of material can be formed from at least one material selected from a group consisting of: a polymeric material, a fluorocarbon material (e.g., polytetrafluoroethylene (PTFE)), silicone, enamel, a ceramic material, and inorganic lubricant material (e.g., titanium nitride, zirconium nitride, titanium aluminum nitride, and nitron). The outer layer of material can help to, for example, inhibit eschar build-up and/or focus the electrosurgical energy to one or more portions of the electrosurgical electrode 128.

In some examples, the first end effector 254A and/or the second end effector 254B of the electrosurgical electrode 128 can additionally include an intermediate layer between the electrosurgical substrate and the outer layer. The intermediate layer can be configured to provide thermal conductivity to help mitigate heating of the outer layer leading to a breakdown of the outer layer. The intermediate layer can also be configured to maintain the electrical conductivity of the electrosurgical substrate such that the intermediate layer does not degrade the transmission of the electrosurgical energy from the electrosurgical substrate to the target tissue.

The intermediate layer can be an anisotropic thermally conductive material, whereby the in-plane (e.g., parallel to the electrode surface) thermal conductivity substantially exceeds the out-of-plane (e.g., perpendicular to the electrode surface) thermal conductivity. The anisotropic thermally conductive material having a coefficient of thermal expansion matched (or approximately 10% greater or approximately 10% lower) to the electrosurgical substrate and outer layer. As an example, this intermediate layer can include at least one material selected from a group consisting of: pyrolytic graphite/carbon, graphene, and Molybdenum disulfide.

As shown in FIG. 2, the electrosurgical electrode can include an electrical contact 258 that can receive electrosurgical energy from the electrosurgical device 112 (e.g., via the housing conductor(s) 134). The electrical contact 258 can be electrically coupled to the first end effector 254A and/or the second end effector 254B such that the electrical contact 258 can provide the electrosurgical energy received from the housing conductor(s) 134 to the first end effector 254A and/or the second end effector 254B to at least one of cut, coagulate, or seal tissue in a monopolar electrosurgical operation.

The electrical contact 258 can be at the intermediate portion 252C of the electrosurgical electrode 128 between the first end 252A and the second end 252B. As described above, the mount 256 can also be at the intermediate portion 252C between the first end 252A and the second end 252B. This can provide for common connection points for (i) mechanically coupling the electrosurgical electrode 128 to the housing 123, and (ii) electrically coupling the electrosurgical electrode 128 to the housing conductor(s) 134 in both the first state and the second state.

In one example, the electrical contact 258 can be configured to simultaneously supply the electrosurgical energy to both the first end effector 254A and the second end effector 254B. For instance, the electrosurgical substrate of the first end effector 254A and the second end effector 254B can be formed as a single, monolithic structure extending along the first end 252A, the intermediate portion 252C, and the second end 252B. The electrical contact 258 can electrically couple to the electrosurgical substrate at a position along the intermediate portion 252C.

In another example, the electrical contact 258 can be configured to supply the electrosurgical energy to only one of the first end effector 254A or the second end effector 254B at a time. For instance, the electrosurgical substrate can include a first portion at the first end effector 254A and a second portion at the second end effector 254B, and an electrode insulator can be between the first portion of the electrosurgical substrate and the second portion of the electrosurgical substrate. The electrode insulator can be configured to resist or prevent electrical communication between the first end effector 254A and the second end effector 254B. The electrical contact 258 can include a first electrical contact coupled to the first portion of the electrosurgical substrate, and a second electrical contact coupled to the second portion of the electrosurgical substrate. When the electrosurgical electrode 128 is coupled to the housing 123 in the first state, the second electrical contact is electrically coupled to the housing conductor(s) 134 and the first electrical contact is decoupled from the housing conductor(s) 134. As such, in the first state, the housing conductor(s) 134 can deliver the electrosurgical energy to the second end effector 254B, and not the first end effector 254A. By contrast, when the electrosurgical electrode 128 is coupled to the housing 123 in the second state, the first electrical contact is electrically coupled to the housing conductor(s) 134 and the second electrical contact is decoupled from the housing conductor(s) 134. As such, in the second state, the housing conductor(s) 134 can deliver the electrosurgical energy to the first end effector 254A, and not the second end effector 254B.

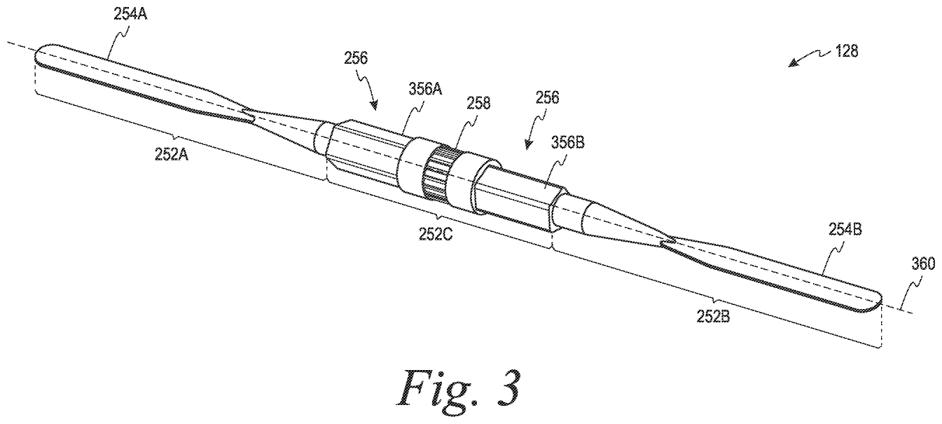

FIG. 3 depicts a perspective view of the electrosurgical electrode 128 shown in FIG. 2, according to one example. As shown in FIG. 3, the electrosurgical electrode 128 includes the first end effector 254A at the first end 252A and the second end effector 254B at the second end 252B. Additionally, as shown in FIG. 3, the first end 252A is opposite the second end 252B, and the electrosurgical electrode 128 has a longitudinal axis 360 extending between the first end 252A and the second end 252B.

In FIG. 3, the first end effector 254A and the second end effector 254B are blade type electrodes, which have the same size, shape, and material composition. In other examples, as described above, the first end effector 254A can have a first configuration, the second end effector 254B has a second configuration, and the first configuration and the second configuration can be different from each other in at least one property selected from a group of properties consisting of: a size, a shape, and a material.

In the example shown in FIG. 3, the mount 256 and the electrical contact 258 are at the intermediate portion 256C, which is between the first end 252A and the second end 252B. In this example, the mount 256 includes a first mount portion 356A on a first side of the electrical contact 258 and a second mount portion 356B on a second side of the electrical contact 258. The first mount portion 356A can couple the electrosurgical electrode 128 to the receptacle of the housing 123 when the electrosurgical electrode 128 is coupled to the housing 123 in the first state (e.g., with the first end effector 254A in the interior cavity of the housing 123 and the second end effector 254B extending distally of the housing 123). The second mount portion 356B can couple the electrosurgical electrode 128 to the receptacle of the housing 123 when the electrosurgical electrode 128 is coupled to the housing 123 in the second state (e.g., with the second end effector 254B in the interior cavity of the housing 123 and the first end effector 254A extending distally of the housing 123).

In FIG. 3, the first mount portion 356A and the second mount portion 356B each have a hexagonal shape that is configured to couple to the receptacle of the housing 123, which has a corresponding hexagonal shape. The mount 256 and the receptacle can have other shapes in other examples.

When the electrosurgical electrode 128 is coupled to the housing 123 in either the first state or the second state, the electrical contact 258 can couple to the housing conductor(s) 134. In FIG. 3, the electrical contact 258 is an annular ring that extends around an entire circumference of the electrosurgical electrode 128. This can facilitate electrically coupling the electrical contact 258 to the housing conductor(s) 134 in a plurality of rotational positions of the electrosurgical electrode 128 relative to the housing 123.

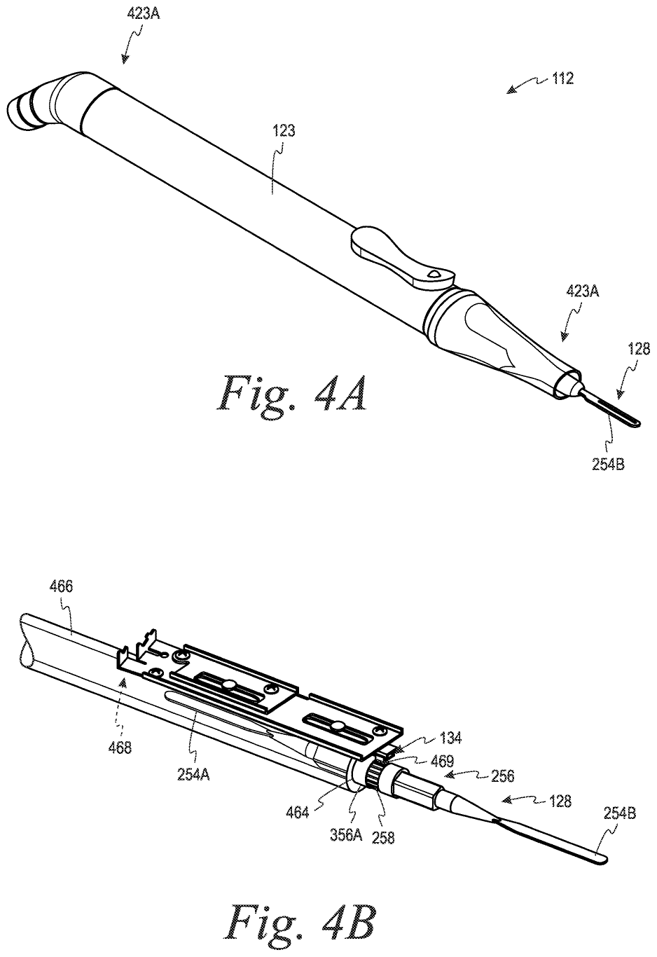

FIG. 4A depicts an implementation of the electrosurgical device 112 shown in FIG. 1 with the electrosurgical electrode 128 shown in FIG. 3 in the first state, FIG. 4B depicts an assembly coupling of the electrosurgical electrode 128 to a receptacle 462 and a housing conductor 434 in the first state, FIG. 4C depicts an implementation of the electrosurgical device 112 shown in FIG. 1 with the electrosurgical electrode 128 shown in FIG. 3 in the second state, FIG. 4D depicts an assembly coupling of the electrosurgical electrode 128 to the receptacle 462 and a housing conductor 434 in the second state.

As shown in FIGS. 4A and 4C, the electrosurgical device 112 includes the housing 123 extending from a proximal end 423A to a distal end 423B, and the electrosurgical electrode 128 extending from the distal end 423B of the housing 123. As shown in FIG. 4A, when the electrosurgical electrode 128 is coupled to the housing 123 in the first state, the first end effector 254A is in the interior cavity of the housing 123 and the second end effector 254B extends from the distal end 423B of the housing 123. As shown in FIG. 4C, when the electrosurgical electrode 128 is coupled to the housing 123 in the second state, the second end effector 254B is in the interior cavity of the housing 123 and the first end effector 254A extends from the distal end 423B of the housing 123.

As shown in FIGS. 4B and 4D, the mount 256 is between the first end effector 254 A and the second end effector 254B, and the mount 256 releasably and reversibly couples the electrosurgical electrode 128 to the housing 123. More particularly, the first mount portion 356A of the mount 256 is coupled to a receptacle 464 of the housing 123 in the first state shown in FIG. 4B, and the second mount portion 356B of the mount 256 is coupled to the receptacle 464 of the housing 123 in the second state shown in FIG. 4D. Also, in FIG. 4B and FIG. 4D, in a plane that is orthogonal to the longitudinal axis (shown in FIG. 3) of the electrosurgical electrode 128, the mount 256 has a cross-sectional shape that is non-circular, and the receptacle 464 has a cross-sectional shape that matches the cross-sectional shape of the mount 256 such that mount 256 is configured to non-rotationally couple to the receptacle 464. In this example, the first mount portion 356A, the second mount portion 356B, and the receptacle 464 are hexagon shaped. However, the first mount portion 356A, the second mount portion 356B, and the receptacle 464 can have other non-circular shapes, or a circular shape in other examples.

In the examples shown in FIG. 4B and FIG. 4D, the housing 123 includes an insulator 466 defining an insulator cavity 468. For example, the insulator 466 can be in the shape of a tube. The insulator 466 can have a length from a proximal end of the insulator 466 to a distal end of the insulator 466. The distal end of the insulator 466 can include the receptacle 464. As shown in FIG. 4B, when the electrosurgical electrode 128 is coupled to the housing 123 in the first state, the first end effector 254A is received in the insulator cavity 468 defined by the insulator 466. The insulator 466 can help to reduce or mitigate transmission of the electrosurgical energy from the first end effector 254A to other components of the electrosurgical device 112 when the electrosurgical electrode 128 is coupled to the housing 123 in the first state. As shown in FIG. 4D, when the electrosurgical electrode 128 is coupled to the housing 123 in the second state, the second end effector 254B is received in the insulator cavity 468 defined by the insulator 466. The insulator 466 can help to reduce or mitigate transmission of the electrosurgical energy from the second end effector 254B to other components of the electrosurgical device 112 when the electrosurgical electrode 128 is coupled to the housing 123 in the second state. In the examples shown in FIG. 4B and FIG. 4D, the length of the insulator 466 is such that the proximal end of the insulator 466 is proximal of the electrosurgical electrode 128.

As shown in FIG. 4B and FIG. 4D, the electrosurgical electrode 128 includes the electrical contact 258 between the first end effector 254A and the second end effector 254B. The housing conductor 134 includes a protrusion 469 that is biased towards the electrical contact 258 to electrically couple the one or more housing conductors 134 to the electrosurgical electrode 128. Although the electrical contact 258 has a ring shape that extends around an entirety of a circumference of the electrosurgical electrode 128 in FIG. 4B and FIG. 4D, the electrical contact 258 can extend around a segment of a circumference of the electrosurgical electrode 128, and the segment is less than an entirety of the circumference.

As described above, in some implementations, the electrosurgical electrode 128 can additionally be rotatable about an axis of rotation that is parallel to the longitudinal axis of the electrosurgical device 112. In one example, the electrosurgical electrode 128 can be rotated by (i) decoupling the mount 256 from the receptacle 464, (ii) after decoupling the mount 256 from the receptacle 464, rotating the electrosurgical electrode 128 relative to the housing 123, and (iii) after rotating the electrosurgical electrode 128 relative the housing 123, recoupling the mount 256 of the electrosurgical electrode 128 to the receptacle 464. As such, in this example, rotating the electrosurgical electrode 128 relative to the housing 123 involves a time consuming process that includes using two hands to manually decouple, rotate, and recouple the electrosurgical electrode 128 while stopping an electrosurgical procedure.

In another example, the electrosurgical device 112 is configured to rotate the electrosurgical electrode 128 relative the housing 123 while the electrosurgical electrode 128 remains coupled to the housing 123 (e.g., while the mount 256 remains coupled to the receptacle 464). This can provide for quicker and more efficient rotation of the electrosurgical electrode 128 and, in some implementations, can provide for rotation of the electrosurgical electrode 128 using a single hand and without removing the hand from the housing 123.

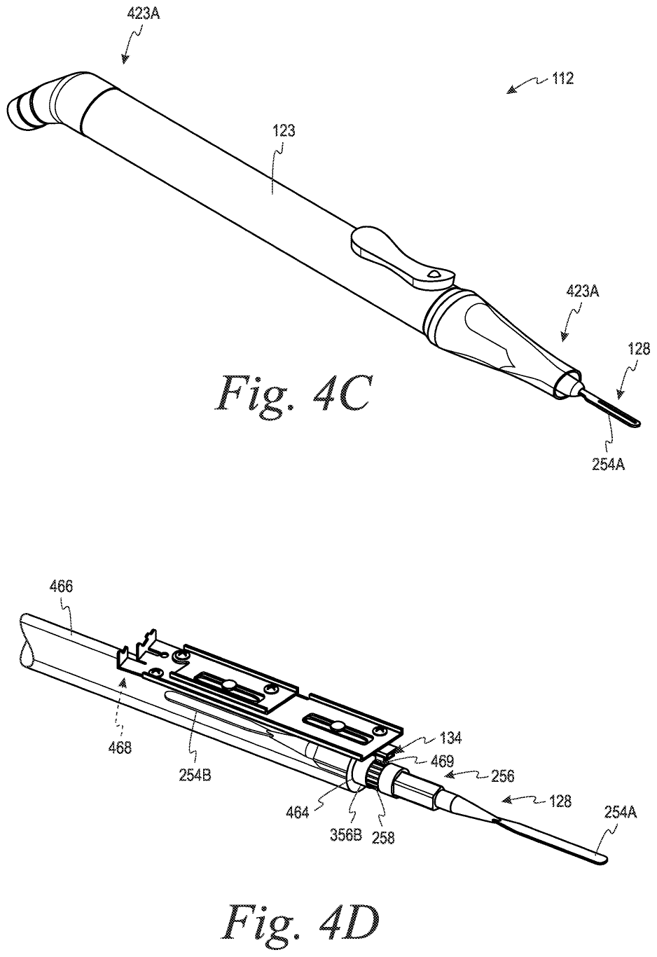

FIG. 5 depicts a simplified block diagram of an electrosurgical system 500, according to an example in which the electrosurgical electrode 128 is rotatable relative to the housing 123 while the electrosurgical electrode 128 is coupled to the housing 123. The electrosurgical system 500 is substantially similar or identical to the electrosurgical system 100 described above with respect to FIG. 1, except the electrosurgical device 112 includes a rotatable member 570 that is operable to rotate the electrosurgical electrode 128 relative to the housing 123.

In FIG. 5, the rotatable member 570 is exposed on an exterior surface of the housing 123 between the proximal end and the distal end of the housing 123 (e.g., the proximal end 423A and the distal end 423B shown in FIGS. 4A and 4C). The rotatable member 570 is rotatable about a longitudinal axis of the housing 123 to rotate the electrosurgical electrode 128 relative to the housing 123.

For instance, in FIG. 5, the rotatable member 570 can be coupled to the receptacle 462 in a rotationally fixed manner such that rotation of the rotatable member 570 relative to the housing 123 causes the receptacle 462 to rotate relative to the housing 123. As described above, the electrosurgical electrode 128 can be releasably or permanently coupled to the receptacle 462 in a rotationally fixed manner such that rotation of the receptacle 462 relative to the housing 123 causes the electrosurgical electrode 128 to rotate relative to the housing 123. In this arrangement, rotating the rotatable member 570 relative to the housing 123 causes the receptacle 462 to rotate relative to the housing 123, which in turn causes the electrosurgical electrode 128 to rotate relative to the housing 123.

In some examples, the rotatable member 570 can be rotatable by greater than 360 degrees relative to the housing. This can improve an ease of use by allowing an operator to use the rotatable member 570 to freely rotate the electrosurgical electrode 128 without limitation. However, in other implementations, the electrosurgical electrode 128 can be rotatable by less than or equal to 360 degrees (e.g., rotatable by 180 degrees, rotatable by 270 degrees, or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement. In one implementation, the rotatable member 570 can be rotatable by no more than approximately 270 degrees. This can provide a good balance of simplified manufacturing and assembly while still allowing the operator to easily rotate the electrosurgical electrode 128 to a desired rotational orientation relative to the housing 123 and/or a surgical site.

In some examples, the rotatable member 570 can extend around an entire circumference of the housing 123. This can beneficially provide greater access to the rotatable member 570 and/or facilitate rotating the rotatable member 570 by equal to or greater than 360 degrees of rotation relative to the housing 123. In examples in which the rotatable member 570 extends around an entire circumference of the housing 123, the rotatable member 570 can include an annular ring. In other examples, the rotatable member 570 can extend around less than an entire circumference of the housing 123. This may be beneficial in implementations in which the rotatable member 570 is rotatable by less than 360 degrees relative to the housing 123. In some examples in which the rotatable member 570 extends around less than the entire circumference of the housing 123, the rotatable member 570 can be an arc shaped structure having a radius of curvature that is approximately equal to a radius of curvature of a surface of the housing 123.

In some examples, the rotatable member 570 is bi-directionally rotatable relative to the housing 123 to rotate the electrosurgical electrode 128 in a first direction and a second direction, and the first direction is opposite the second direction. This can allow the operator to more quickly rotate the electrosurgical electrode 128 to a desired rotational orientation relative to the housing 123 and/or the surgical site. In other examples, the rotatable member 570 can be rotatable in the first direction relative to the housing 123 and not rotatable in the second direction relative to the housing 123. This may be beneficial to resist inadvertent rotation of the rotatable member 570 in an implementation in which the rotatable member 570 is positioned on the handle 124 at a location where the operator normally grasps the electrosurgical device 112 and applies a force to the rotatable member 570 in the second direction while cutting or coagulating tissue at the surgical site.

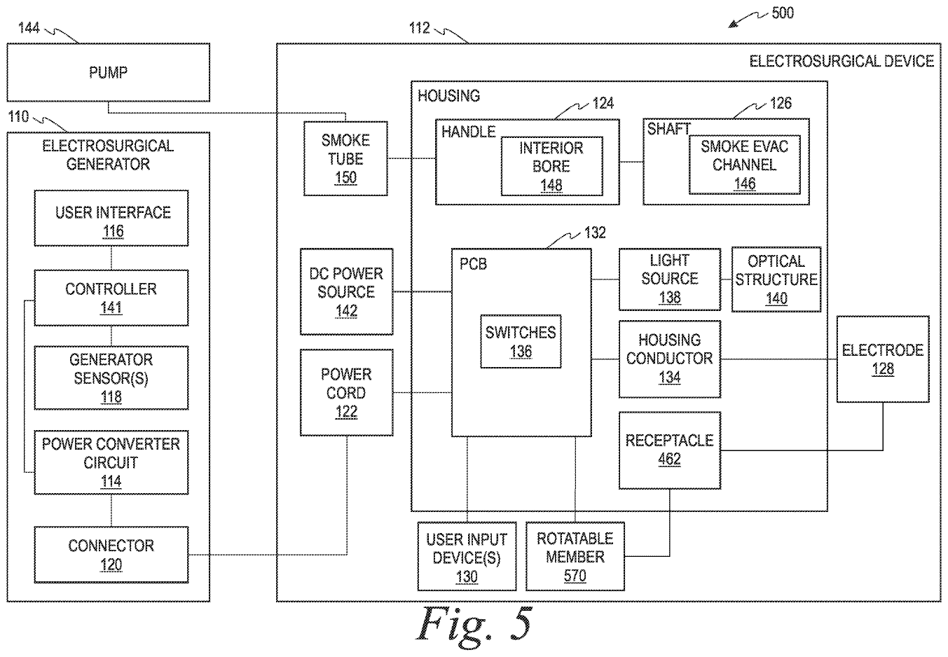

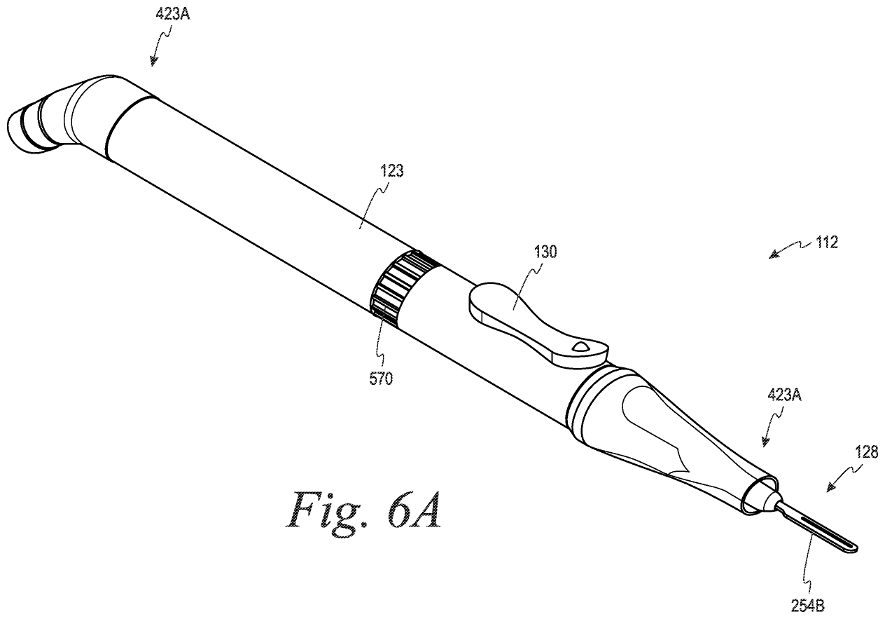

FIG. 6A-6D depict an implementation of the electrosurgical device 112 of FIG. 5 in which the electrosurgical electrode 128 is rotatable relative to the housing 123 while the electrosurgical electrode 128 is coupled to the housing 123, according to an example. In this example, the electrosurgical electrode 128 is configured as described above with respect to FIGS. 2-4D. As such, the electrosurgical electrode 128 has the first end effector 254A at the first end 252A of the electrosurgical electrode 128 and the second end effector 254B at the second end 252B of the electrosurgical electrode 128. As described below with respect to FIGS. 7A-7B, in other examples, the electrosurgical electrode 128 can have a single end effector at one end and the mount 256 can be at the opposing end of the electrosurgical electrode 128.

FIG. 6A depicts an implementation of the electrosurgical device 112 shown in FIG. 5 with the electrosurgical electrode 128 shown in FIG. 3 in the first state, according to an example. FIG. 6B depicts an assembly of the rotatable member 570, the receptacle 462, the electrosurgical electrode 128 coupled to the receptacle 462 in the first state, and the housing conductor 434 according an example for the implementation shown in FIG. 6A, according to an example. FIG. 6C depicts an implementation of the electrosurgical device 112 shown in FIG. 5 with the electrosurgical electrode 128 shown in FIG. 3 in the second state, according to an example. FIG. 6D depicts an assembly of the rotatable member 570, the receptacle 462, the electrosurgical electrode 128 coupled to the receptacle 462 in the second state, and the housing conductor 434 according an example for the implementation shown in FIG. 6C, according to an example.

As shown in FIG. 6A and FIG. 6C, the electrosurgical device 112 includes the housing 123 extending from the proximal end 423A to the distal end 423B, and the electrosurgical electrode 128 extending from the distal end 423B of the housing 123. As shown in FIG. 6A, when the electrosurgical electrode 128 is coupled to the housing 123 in the first state, the first end effector 254A is in the interior cavity of the housing 123 and the second end effector 254B extends from the distal end 423B of the housing 123. As shown in FIG. 6C, when the electrosurgical electrode 128 is coupled to the housing 123 in the second state, the second end effector 254B is in the interior cavity of the housing 123 and the first end effector 254A extends from the distal end 423B of the housing 123.

As shown in FIGS. 6B and 6D, the mount 256 is between the first end effector 254 A and the second end effector 254B, and the mount 256 releasably and reversibly couples the electrosurgical electrode 128 to the housing 123. More particularly, the first mount portion 356A of the mount 256 is coupled to a receptacle 464 of the housing 123 in the first state shown in FIG. 6B, and the second mount portion 356B of the mount 256 is coupled to the receptacle 464 of the housing 123 in the second state shown in FIG. 6D. Also, in FIG. 6B and FIG. 6D, in a plane that is orthogonal to the longitudinal axis (shown in FIG. 3) of the electrosurgical electrode 128, the mount 256 has a cross-sectional shape that is non-circular, and the receptacle 464 has a cross-sectional shape that matches the cross-sectional shape of the mount 256 such that mount 256 is configured to non-rotationally couple to the receptacle 464. In this example, the first mount portion 356A, the second mount portion 356B, and the receptacle 464 are hexagon shaped. However, the first mount portion 356A, the second mount portion 356B, and the receptacle 464 can have other non-circular shapes in other examples.

In the example shown in FIG. 6B and FIG. 6D, the housing 123 includes the insulator 466 defining the insulator cavity 468. The insulator 466 can couple the rotatable member 570 to the receptacle 464 such that the rotatable member 570 is rotationally fixed relative to the receptacle 464. For instance, in the example shown in FIG. 6B and FIG. 6D, the receptacle 464 can coupled to or defined by the distal end of the insulator 466, and the rotatable member 570 can be coupled to the proximal end of the insulator 466. In this arrangement, rotation of the rotatable member 570 relative to the housing 123 causes (i) the insulator 466 to rotate due to the coupling between the insulator 466 and the rotatable member 570, (ii) the receptacle 464 to rotate due to the rotation of the insulator 466, and (iii) the electrosurgical electrode 128 to rotate due to the coupling between the mount 256 and the receptacle 464.

As shown in FIG. 6B and FIG. 6D, the electrosurgical electrode 128 includes the electrical contact 258, and the housing conductor 134 includes a protrusion 469 that is biased towards the electrical contact 258 to electrically couple the one or more housing conductors 134 to the electrosurgical electrode 128. The protrusion 469 and the electrical contact 258 are in the interior cavity defined by the housing 123. Additionally, in this example, the protrusion 469 and the electrical contact 258 are distal of the rotatable member 570. This can help to provide for a relatively shorter electrosurgical electrode 128. In another example, the protrusion 469 and the electrical contact 258 can be proximal of the rotatable member 470. This arrangement may be beneficial in an implementation in which the electrosurgical electrode 128 is relatively long.

As described above, the electrical contact 258 can have a ring shape that extends around an entirety of a circumference of the electrosurgical electrode 128, or the electrical contact 258 can extend around a segment of a circumference of the electrosurgical electrode 128, and the segment is less than an entirety of the circumference. For instance, in the example shown in FIGS. 6A-6D, the rotatable member 570 can be rotatable by greater than or equal to 360 degrees relative to the housing 123 and the ring shape of the electrical contact 258 can help to maintain electrical communication between the electrosurgical electrode 128 and the housing conductor(s) 134 in all rotational positions of the electrosurgical electrode 128 relative to the housing 123. However, in an example in which the rotatable member 570 is rotatable by less than 360 degrees, the electrical contact 258 can extend around the segment of the circumference that is sufficient to maintain the electrical communication between the electrosurgical electrode 128 and the housing conductor(s) 134 in all rotational positions of the electrosurgical electrode 128 relative to the housing 123.

Within examples, the electrosurgical device 112 can be configured to resist inadvertent rotation of the rotatable member 570 and the electrosurgical electrode 128. For instance, the electrical contact 258 can a plurality of detents 672 around at least a portion of a circumference of the electrosurgical electrode 128, and the protrusion 469 can be configured to engage the plurality of detents 672, one detent 672 at a time, to provide a force to the electrosurgical electrode 128 that opposes a rotational force applied to the rotatable member 570. In the example shown in FIG. 6B and FIG. 6D, the detents 672 include a plurality of teeth 674 that extend outwardly from a base surface 676 of the electrical contact 258. A gap between each adjacent pair of the teeth 674 corresponds to a respective rotational position of the electrosurgical electrode 128 relative to the housing 123. At each rotational position, the protrusion 469 is configured to be received in the gap between the adjacent ones of the teeth 674 corresponding to the rotational position. In this example, the teeth 674 can engage the protrusion 469 to help resist rotation of the electrosurgical electrode 128 when a force less than a threshold amount of force is applied to the rotational member 570 and/or the electrosurgical electrode 128 (e.g., to mitigate inadvertent rotation), and the protrusion 469 can be configured to move over the teeth 674 when a force that is greater than threshold amount of force is applied to the rotatable member 570 (e.g., to permit intentional rotation of the electrosurgical electrode 128).

FIGS. 7A-7B depict implementation of the electrosurgical device 112 shown in FIG. 5, according to another example. The electrosurgical device 112 shown in FIGS. 7A-7B is substantially similar or identical to the electrosurgical device shown in FIGS. 6A-6D, except the rotatable member 570 is shown at an alternative location on the housing 123 and the detents 672 have an alternative configuration.

As shown in FIG. 7A, the electrosurgical device 112 includes the user input device 130, which is operable to supply electrosurgical energy to the electrosurgical electrode 128. Additionally, as shown in FIG. 7A, the user input device 130 extends over at least a portion of the rotatable member 570. In this arrangement, the operator can easily operate both the user input device 130 and the rotatable member 570 with a single hand (and without removing the hand from the housing 123). Additionally or alternatively, for example, positioning a portion of the rotatable member 570 under the user input device 130 can help to mitigate inadvertent rotation of the rotatable member 570 while performing electrosurgery as the operator's finger(s) may be position on the user input device 130 while the rest of the operator's hand grasps a portion of the housing 123 that is proximal of the user input device 130 (and the rotatable member 570).

As shown in FIG. 7B, in this example, the rotatable member 570 can be coupled to a central portion of the insulator 466 between the proximal end of the insulator 466 and the distal end of the insulator 466 (e.g., at a position located between the insulator 466 and the stamped metal component 730 of the user input device 130 shown in FIG. 6B and FIG. 6D). This can provide for positioning the rotatable member 570 closer to the distal end 423B of the housing 123 while allowing the insulator 466 to have a length such that the proximal end of the insulator 466 is proximal of the electrosurgical electrode 128.

As also shown in FIG. 7B, in this example, the detents 672 can include a plurality of recesses 778 that extend inwardly from the base surface 676 of the electrical contact 258. Each recess 778 corresponds to a respective rotational position of the electrosurgical electrode 128 relative to the housing 123. At each rotational position, the protrusion 469 is configured to be received in the recess 778 corresponding to the rotational position. In this example, the recesses 778 can engage the protrusion 469 to help resist rotation of the electrosurgical electrode 128 when a force less than a threshold amount of force is applied to the rotational member 570 and/or the electrosurgical electrode 128 (e.g., to mitigate inadvertent rotation), and the protrusion 469 can be configured to move out of the recesses 778 when a force that is greater than threshold amount of force is applied to the rotatable member 570 (e.g., to permit intentional rotation of the electrosurgical electrode 128).

In the examples shown in FIGS. 6A-7B, the rotatable member 570 extends around the entire circumference of the housing 123 and the electrical contact 258 extends around the entire circumference of the electrosurgical electrode 128. As described above, in other examples, the rotatable member 570 can extend around less than the entire circumference of the housing 123, and/or the electrical contact 258 can extend around less than the entire circumference of the electrosurgical electrode 128.

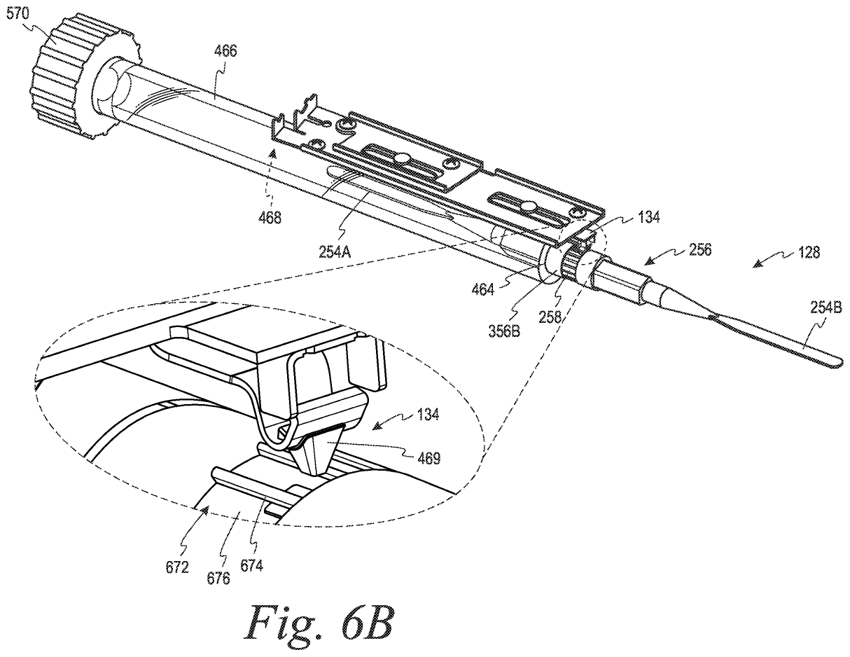

FIGS. 8A-8B depict an implementation of the electrosurgical device 112 shown in FIG. 5 according to an example in which the rotatable member 570 extends around less than the entire circumference of the housing 123, and the electrical contact 258 extends around less than the entire circumference of the electrosurgical electrode 128, according to one example. In this example, the rotatable member 570 and the electrosurgical electrode 128 can be rotatable by no more than 270 degrees. As shown in FIGS. 8A-8B, the rotatable member 570 is an arc shaped structure having a radius of curvature that is approximately equal to a radius of curvature of a surface of the housing 123, and the electrical contact 270 extends around approximately 270 degrees of the circumference of the electrosurgical electrode 128.

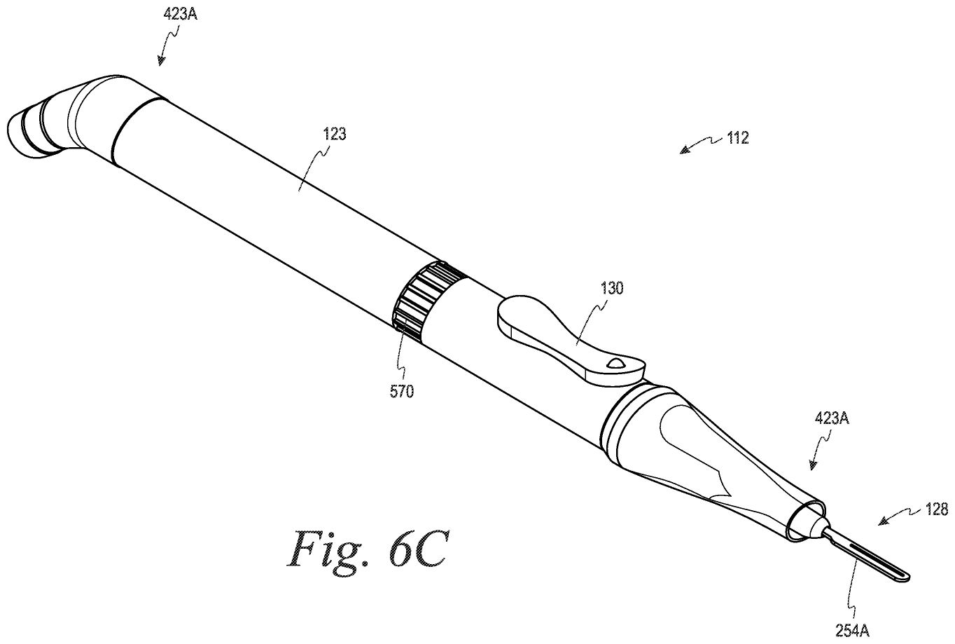

FIGS. 9A-9E depict a plurality of electrosurgical electrodes 128 that can be used with the electrosurgical device 112 shown in FIG. 1 and/or FIG. 5, according to additional examples. FIG. 9A depicts the electrosurgical electrode 128 having a single end effector 954 (as opposed to the first end effector 254A and the second end effector 254B). As shown in FIG. 9A, the electrosurgical electrode 128 also includes a mount 956 and an electrical contact 958, which are substantially similar or identical to the mount 956 and the electrical contact 258 described above.

As described above with respect to FIG. 2, in some examples, the electrical contact 258 can be configured to supply the electrosurgical energy to only one of the first end effector 254A or the second end effector 254B at a time. FIG. 9B depicts the electrosurgical electrode 128 according one such example. In FIG. 9B, the electrosurgical electrode 128 includes a first electrical contact 958A electrically coupled to the first end effector 254A and a second electrical contact 958B electrically coupled to the second end effector 254B. For instance, the electrosurgical substrate can include a first portion at the first end effector 254A and a second portion at the second end effector 254B, and an electrical insulator can be between the first portion of the electrosurgical substrate and the second portion of the electrosurgical substrate. In FIG. 9B, the first electrical contact 958A and the first end effector 254A are electrically isolated from the second electrical contact 958B and the second end effector 254B by an electrode insulator 980.

When the electrosurgical electrode 128 is coupled to the housing 123 in the first state, the second electrical contact 958B is electrically coupled to the housing conductor(s) 134 and the first electrical contact 958A is decoupled from the housing conductor(s) 134. As such, in the first state, the housing conductor(s) 134 can deliver the electrosurgical energy to the second end effector 254B, and not the first end effector 254A. By contrast, when the electrosurgical electrode 128 is coupled to the housing 123 in the second state, the first electrical contact 958A is electrically coupled to the housing conductor(s) 134 and the second electrical contact 958B is decoupled from the housing conductor(s) 134. As such, in the second state, the housing conductor(s) 134 can deliver the electrosurgical energy to the first end effector 254A, and not the second end effector 254B.

FIG. 9C depicts an implementation of the electrosurgical electrode 128 in which the first end effector 254A has a different size than the second end effector 254B, according to an example. FIG. 9D depicts an implementation of the electrosurgical electrode 128 in which the first end effector 254A and the second end effector 254B are different types of electrodes, according to one example. For instance, in FIG. 9D, the first end effector 254A is a blade type electrode and the second end effector is a ball tip type electrode. FIG. 9D depicts an implementation of the electrosurgical electrode 128 in which the first end effector 254A and the second end effector 254B are different types of electrodes, according to another example. For instance, in FIG. 9D, the first end effector 254A is a blade type electrode and the second end effector is a needle type electrode.

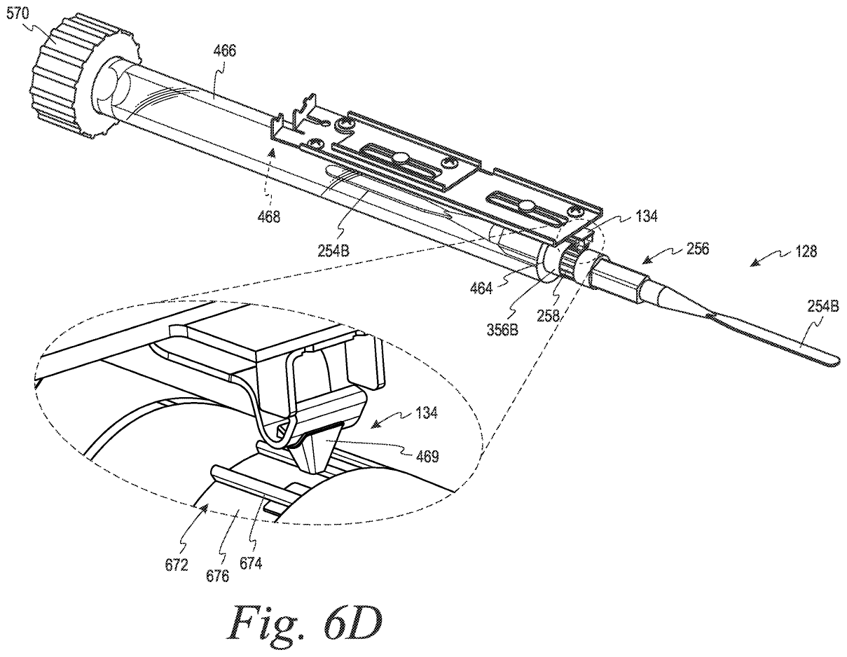

Referring now to FIG. 10, a flowchart for a process 1000 of operating an electrosurgical device is shown according to an example. As shown in FIG. 10, the process 1000 includes providing an electrosurgical device at block 1010. The electrosurgical device includes a housing extending from a proximal end to a distal end, and an electrosurgical electrode coupled to the housing such that the electrosurgical electrode extends from the distal end of the housing. At block 1012, the process 1000 includes rotating a rotatable member relative to the housing. The rotatable member is exposed on an exterior surface of the housing between the proximal end and the distal end of the housing. Responsive to rotating the rotatable member relative to the housing at block 1012, the process 1000 includes rotating the electrosurgical electrode relative the housing at block 1014.

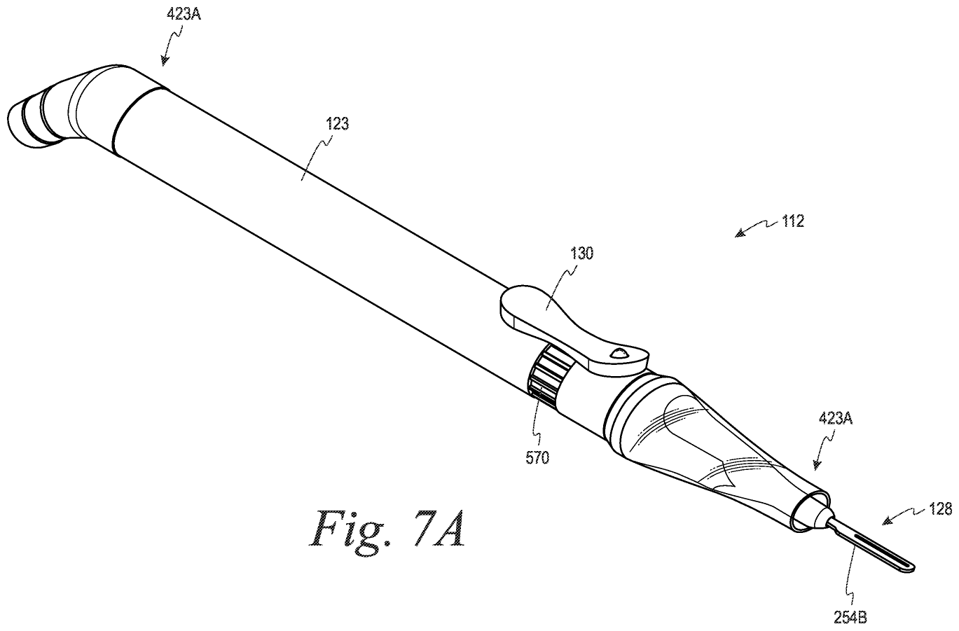

FIGS. 11-17 depict additional aspects of the process 1000 according to further examples. In FIG. 11, the electrosurgical device includes one or more housing conductors that are configured to conduct electrosurgical energy from a power cord to the electrosurgical electrode, the electrosurgical electrode includes an electrical contact, and the one or more housing conductors includes a protrusion. As shown in FIG. 11, the process 1000 further includes biasing the protrusion towards the electrical contact to electrically couple the one or more housing conductors to the electrosurgical electrode at block 1016.

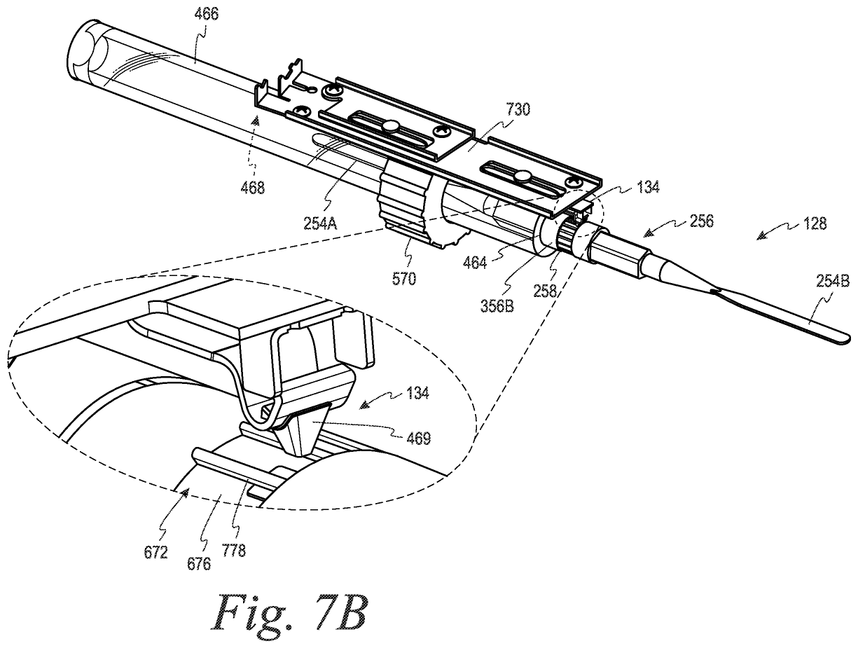

In FIG. 12, the electrical contact includes a plurality of detents around at least a portion of a circumference of the electrosurgical electrode. As shown in FIG. 12, the process 1000 includes providing, by an engagement between the protrusion and the plurality of detents, a force to the electrosurgical electrode that opposes a rotational force applied to the rotatable member at block 1018.

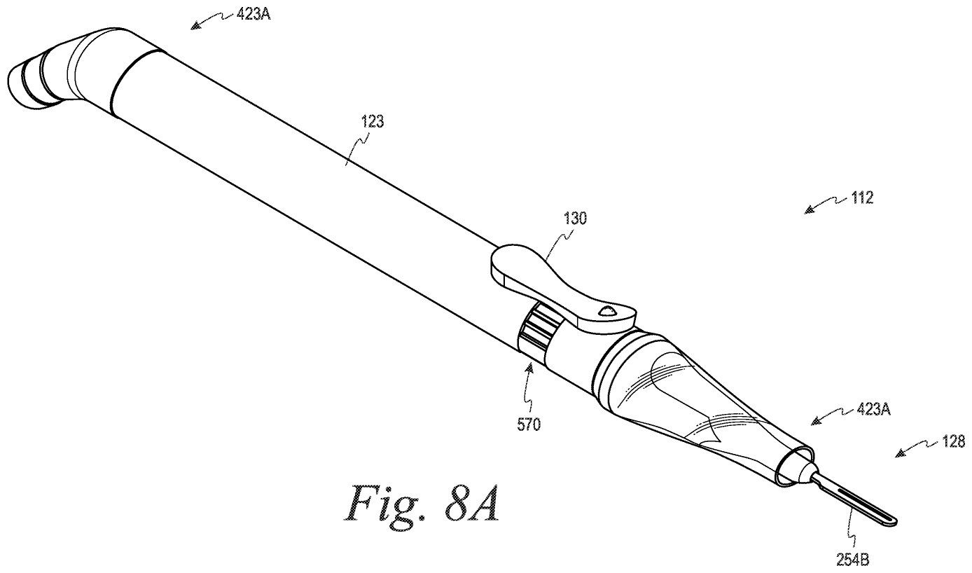

In FIG. 13, rotating the rotatable member at block 1014 includes bi-directionally rotating the rotatable member relative to the housing to rotate the electrosurgical electrode in a first direction and a second direction at block 1020. The first direction is opposite the second direction.

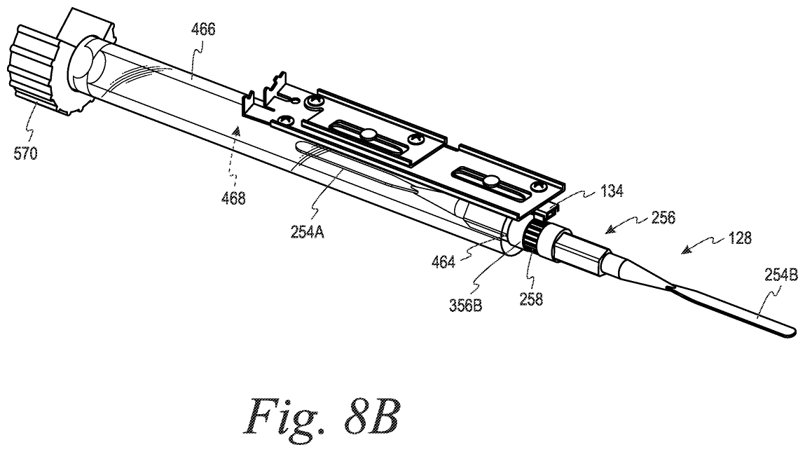

In FIG. 14, rotating the rotatable member at block 1014 includes rotating the rotatable member in a first direction relative to the housing while preventing rotation of the rotatable member in a second direction relative to the housing at block 1022. The first direction is opposite the second direction.

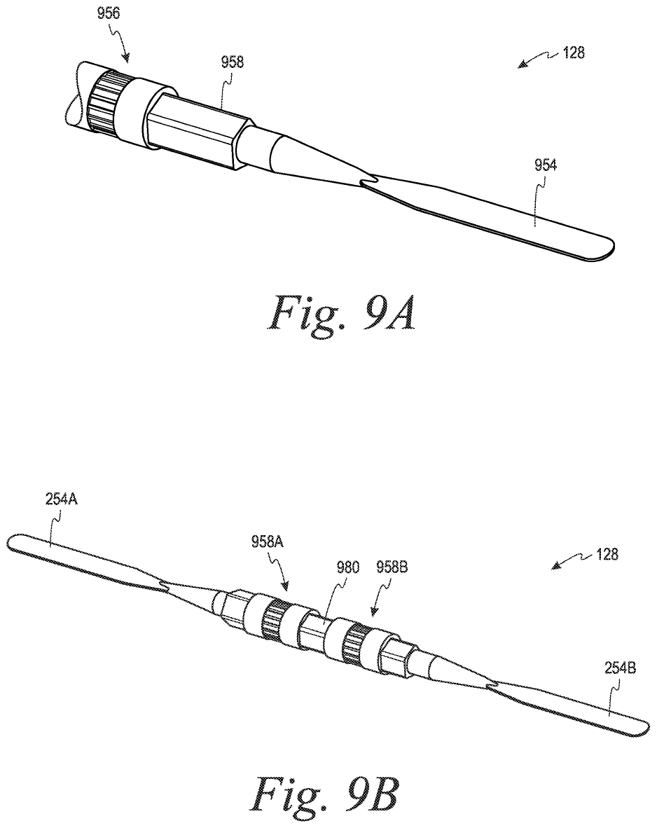

In FIG. 15, the electrosurgical electrode has a longitudinal axis extending between a first end and a second end, and the electrosurgical electrode has a first end effector at the first end and a second end effector at the second end. As shown in FIG. 15, the process 1000 can also include coupling the electrosurgical electrode to the housing in a first state in which the first end effector in an interior cavity of the housing and the second end effector extends from the distal end of the housing at block 1024.

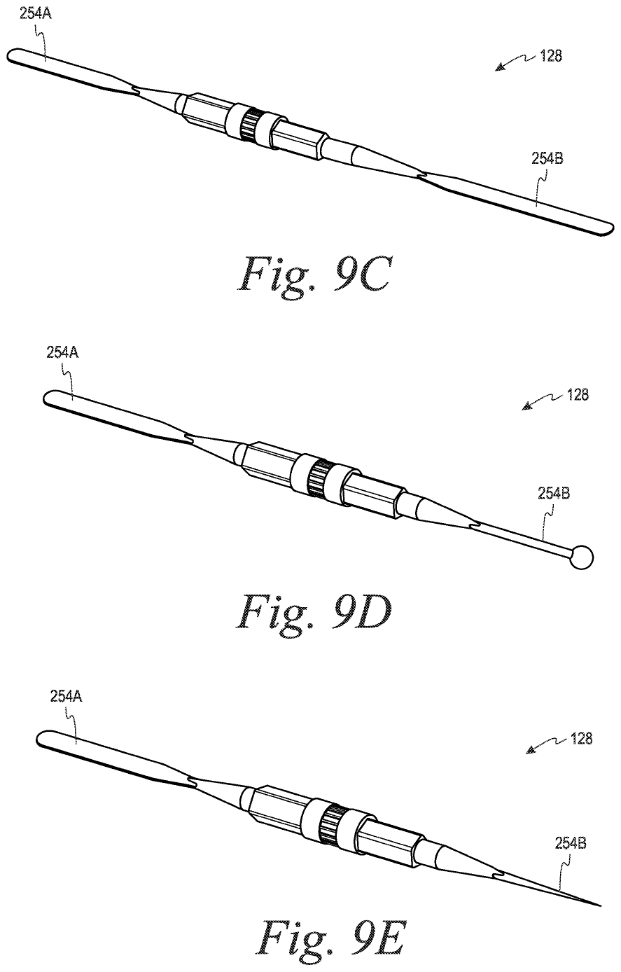

As shown in FIG. 16, after coupling the electrosurgical electrode to the housing in the first state at block 1024, the process 1000 can include (i) decoupling the electrosurgical electrode from the housing at block 1026 and (ii) coupling the electrosurgical electrode to the housing in a second state in which the second end effector is in the interior cavity of the housing and the first end effector extends from the distal end of the housing at block 1028.

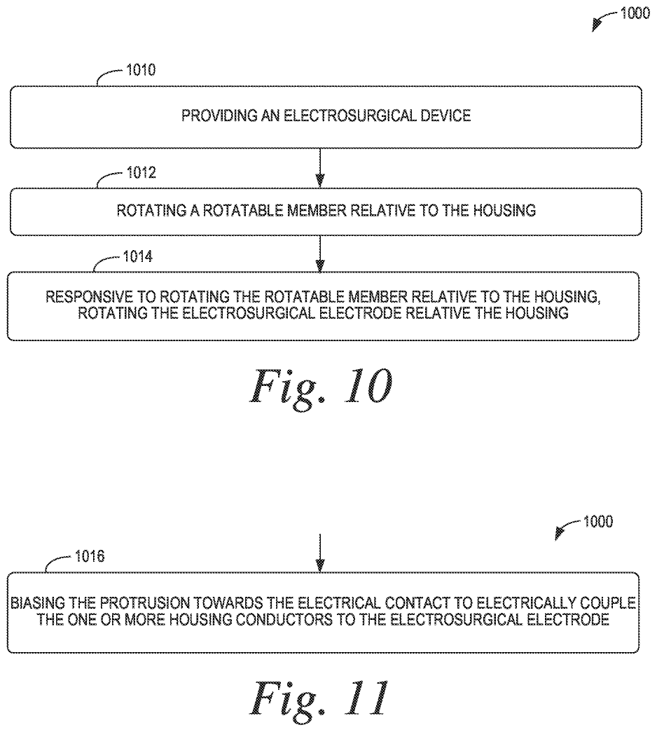

In FIG. 17, the housing includes an insulator defining an insulator cavity, and a receptacle that is coupled to a mount of the electrosurgical electrode. In the first state, the first end effector is received in the insulator cavity. In the second state, the second end effector is received in the insulator cavity. The insulator couples the rotatable member and the receptacle such that the rotatable member is rotationally fixed relative to the receptacle. As shown in FIG. 17, rotating the rotatable member relative to the housing at block 1012 includes rotating the insulator, the receptacle, and the electrosurgical electrode coupled to the receptacle at block 1030.

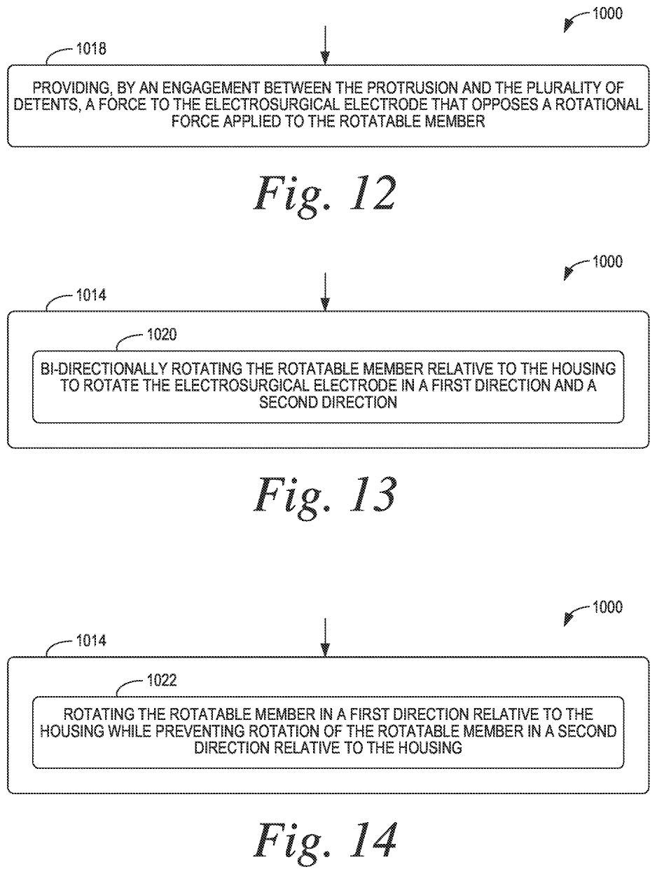

Referring now to FIG. 18, a flowchart for a process 1800 of forming an electrosurgical device is shown according to an example. As shown in FIG. 18, the process 1800 includes forming a housing at block 1810. The housing extends from a proximal end to a distal end. At block 1812, the process 1800 includes coupling an electrosurgical electrode to the housing such that the electrosurgical electrode extends from the distal end of the housing. At block 1814, the process 1800 includes coupling a rotatable member to the housing such that the rotatable member is exposed on an exterior surface of the housing between the proximal end and the distal end of the housing. The rotatable member is rotatable about a longitudinal axis of the housing to rotate the electrosurgical electrode relative the housing.

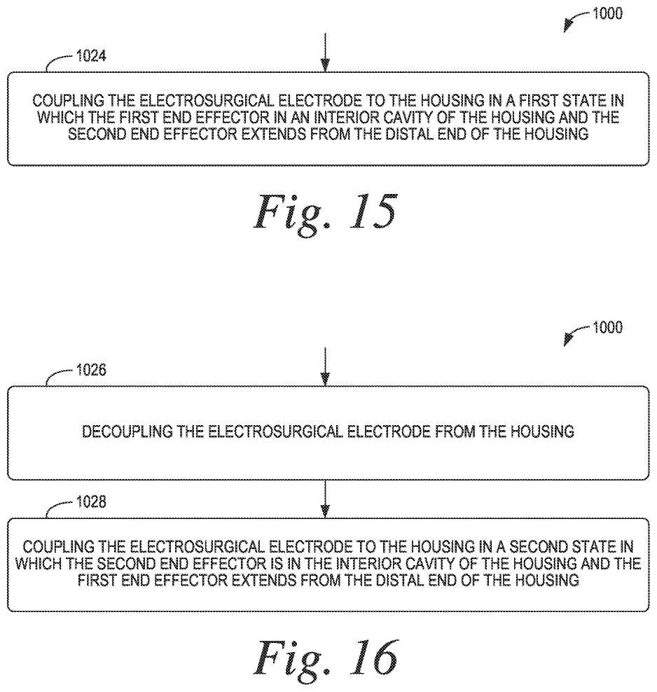

Referring now to FIG. 19, a flowchart for a process 1900 of operating an electrosurgical device is shown according to another example. As shown in FIG. 19, the process 1900 includes providing a housing at block 1910. The housing extends from a proximal end to a distal end. At block 1912, the process 1900 includes coupling an electrosurgical electrode to the housing such that the electrosurgical electrode extending from the distal end of the housing. The electrosurgical electrode extends between a first end and a second end. The electrosurgical electrode has a first end effector at the first end and a second end effector at the second end.

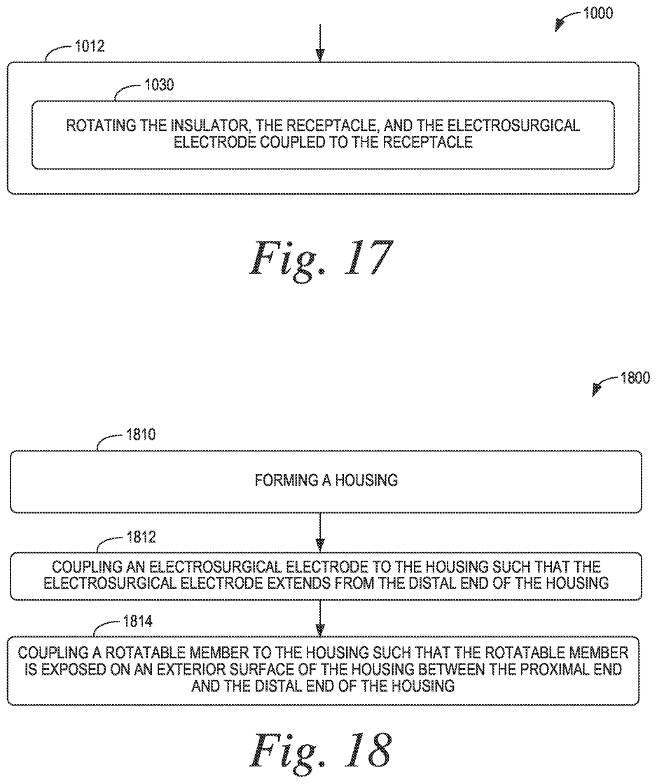

FIGS. 20-24 depict additional aspects of the process 1900 according to further examples. As shown in FIG. 20, coupling the electrosurgical electrode to the housing at block 1912 includes coupling the electrosurgical electrode to the housing in a first state in which the first end effector in an interior cavity of the housing and the second end effector extends from the distal end of the housing at block 1914.

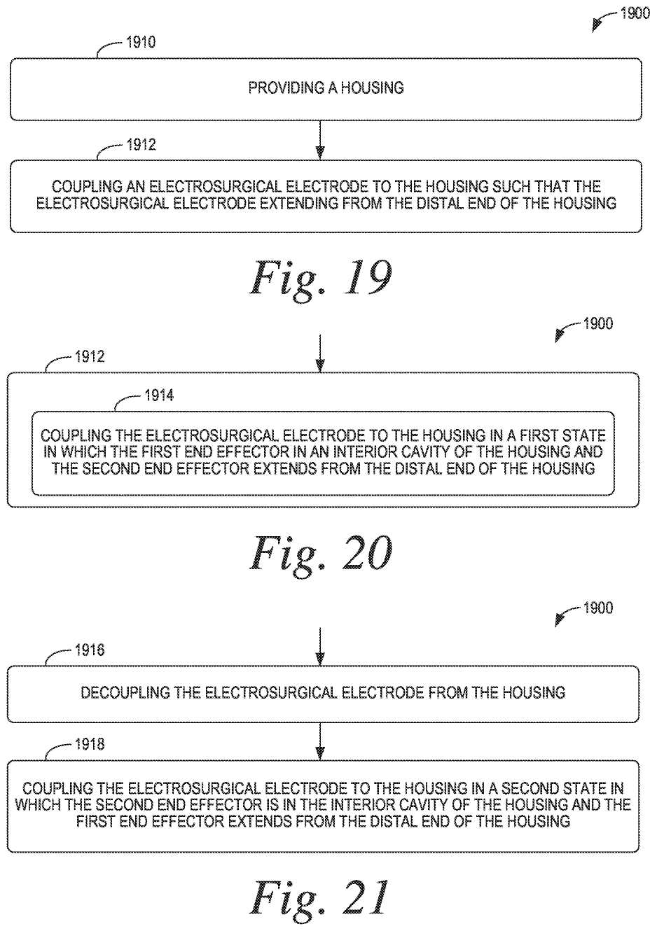

As shown in FIG. 21, after coupling the electrosurgical electrode to the housing in the first state at block 1914, the process 1900 can include (i) decoupling the electrosurgical electrode from the housing at block 1916 and (ii) coupling the electrosurgical electrode to the housing in a second state in which the second end effector is in the interior cavity of the housing and the first end effector extends from the distal end of the housing at block 1918.

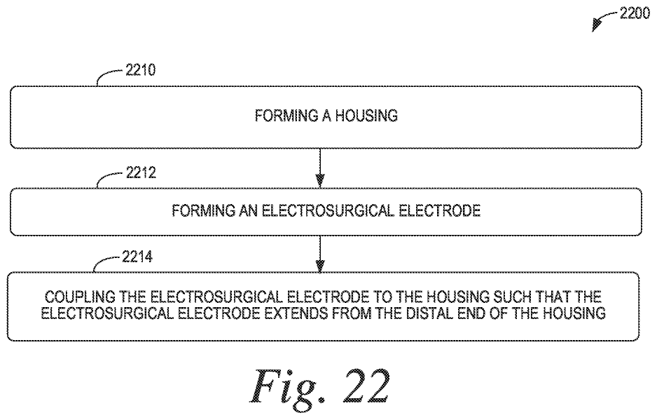

Referring now to FIG. 22, a flowchart for a process 2200 of forming an electrosurgical device is shown according to an example. As shown in FIG. 22, the process 2200 includes forming a housing at block 2210. The housing extends from a proximal end to a distal end. At block 2212, the process 2200 includes forming an electrosurgical electrode. The electrosurgical electrode extends between a first end and a second end. The electrosurgical electrode has a first end effector at the first end and a second end effector at the second end. At block 2214, the process 2200 includes coupling the electrosurgical electrode to the housing such that the electrosurgical electrode extends from the distal end of the housing.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

1. An electrosurgical device, comprising:
a housing extending from a proximal end to a distal end;
an electrosurgical electrode extending from the distal end of the housing; and
a rotatable member exposed on an exterior surface of the housing between the proximal end and the distal end of the housing,
wherein rotatable member is rotatable about a longitudinal axis of the housing to rotate the electrosurgical electrode relative the housing.

2. The electrosurgical device of claim 1, wherein the rotatable member extends around an entire circumference of the housing.

3. The electrosurgical device of claim 1, wherein the rotatable member extends around less than an entire circumference of the housing.

4. The electrosurgical device of claim 1, further comprising one or more housing conductors that are configured to conduct electrosurgical energy from a power cord to the electrosurgical electrode,
wherein the electrosurgical electrode comprises an electrical contact, and
wherein the one or more housing conductors comprises a protrusion that is biased towards the electrical contact to electrically couple the one or more housing conductors to the electrosurgical electrode.

5. The electrosurgical device of claim 4, wherein the electrical contact comprises a plurality of detents around at least a portion of a circumference of the electrosurgical electrode, and
wherein the protrusion is configured to engage the plurality of detents, one detent at a time, to provide a force to the electrosurgical electrode that opposes a rotational force applied to the rotatable member.

6. The electrosurgical device of claim 5, wherein the plurality of detents comprise a plurality of teeth that extend outwardly from a base surface of the electrical contact,
wherein a gap between each adjacent pair of the plurality of teeth corresponds to a respective rotational position of the electrosurgical electrode relative to the housing, and
wherein, at each rotational position, the protrusion is configured to be received in the gap between the adjacent pair of the plurality of teeth corresponding to the rotational position.

7. The electrosurgical device of claim 5, wherein the plurality of detents comprise a plurality of recesses that extend inwardly from a base surface of the electrical contact,
wherein each recess corresponds to a respective rotational position of the electrosurgical electrode relative to the housing, and
wherein, at each rotational position, the protrusion is configured to be received in the recess corresponding to the rotational position.

8. The electrosurgical device of claim 4, wherein the electrical contact has a ring shape that extends around an entirety of a circumference of the electrosurgical electrode.

9. The electrosurgical device of claim 4, wherein the electrical contact extends around a segment of a circumference of the electrosurgical electrode, wherein the segment is less than an entirety of the circumference.

10. The electrosurgical device of claim 4, wherein the protrusion and the electrical contact are distal of the rotatable member.

11. The electrosurgical device of claim 4, wherein the protrusion and the electrical contact are in an interior cavity defined by the housing.

12. The electrosurgical device of claim 1, wherein the rotatable member is bi-directionally rotatable relative to the housing to rotate the electrosurgical electrode in a first direction and a second direction, wherein the first direction is opposite the second direction.

13. The electrosurgical device of claim 1, wherein the rotatable member is rotatable in a first direction relative to the housing and not rotatable in a second direction relative to the housing, wherein the first direction is opposite the second direction.

14. The electrosurgical device of claim 4, wherein the electrosurgical electrode has a longitudinal axis extending between a first end and a second end, and
wherein the electrosurgical electrode has a first end effector at the first end and a second end effector at the second end.

15. The electrosurgical device of claim 14, wherein the electrosurgical electrode comprises an electrical contact between the first end effector and the second end effector.

16. The electrosurgical device of claim 14, wherein the first end effector has a first configuration, and the second end effector has a second configuration,
wherein the first configuration and the second configuration are different from each other in at least one property selected from a group of properties consisting of: a size, a shape, and a material.

17. The electrosurgical device of claim 16, wherein the first end effector is an electrosurgical blade and the second end effector is ball tip type electrode.

18. The electrosurgical device of claim 14, wherein the first end effector has a first configuration, the second end effector has a second configuration,
wherein the first configuration and the second configuration are the same as each other in a size, a shape, and a material.

19. The electrosurgical device of claim 14, wherein the housing comprises:
an insulator defining an insulator cavity; and
a receptacle that is configured to couple to a mount of the electrosurgical electrode,
wherein, in a first state, the first end effector is received in the insulator cavity,
wherein, in a second state, the second end effector is received in the insulator cavity, and
wherein the insulator couples the rotatable member and the receptacle such that the rotatable member is rotationally fixed relative to the receptacle.

20. The electrosurgical device of claim 1, wherein the rotatable member is rotatable by greater than 360 degrees relative to the housing.

21. The electrosurgical device of claim 1, wherein the rotatable member is rotatable by no more than 270 degrees relative to the housing.

22. The electrosurgical device of claim 1, further comprising a user input device that is operable to supply electrosurgical energy to the electrosurgical electrode.

23. The electrosurgical device of claim 22, wherein the user input device extends over at least a portion of the rotatable member.

24. The electrosurgical device of claim 1, wherein the housing defines a smoke evacuation channel in an interior cavity of the housing.

25. An electrosurgical device, comprising:
a housing extending from a proximal end to a distal end; and
an electrosurgical electrode extending from the distal end of the housing,
wherein the electrosurgical electrode extends between a first end and a second end, and
wherein the electrosurgical electrode has a first end effector at the first end and a second end effector at the second end.

26. The electrosurgical device of claim 25, wherein the electrosurgical electrode is configured to releasably couple to the housing in (i) a first state in which the first end effector in an interior cavity of the housing and the second end effector extends from the distal end of the housing, and (ii) a second state in which the second end effector is in the interior cavity of the housing and the first end effector extends from the distal end of the housing.

27. The electrosurgical device of claim 26, wherein the housing comprises an insulator defining an insulator cavity, and
wherein, in the first state, the first end effector is received in the insulator cavity, and
wherein, in the second state, the second end effector is received in the insulator cavity.

28. The electrosurgical device of claim 27, wherein the insulator is a tube.

29. The electrosurgical device of claim 25, wherein the electrosurgical electrode comprises a mount between the first end effector and the second end effector, and
wherein the mount is configured to releasably and reversibly couple the electrosurgical electrode to the housing.

30. The electrosurgical device of claim 29, wherein, in a plane that is orthogonal to a longitudinal axis of the electrosurgical electrode, the mount has a cross-sectional shape that is non-circular.

31. The electrosurgical device of claim 30, wherein the housing comprises a receptacle that is configured to couple to the mount of the electrosurgical electrode, and
wherein, in the plane that is orthogonal to the longitudinal axis, the receptacle has a cross-sectional shape that matches the cross-sectional shape of the mount such that mount is configured to non-rotationally couple to the receptacle.

32. An electrosurgical electrode, comprising:
a first end effector at a first end;
a second end effector at a second end, wherein the first end is opposite the second end, wherein the electrosurgical electrode has a longitudinal axis extending between the first end and the second end; and
a mount between the first end effector and the second end effector, wherein the mount is configured to couple the electrosurgical electrode to a housing of an electrosurgical device.

33. The electrosurgical electrode of claim 32, wherein the electrosurgical electrode comprises an electrical contact between the first end effector and the second end effector.

34. The electrosurgical electrode of claim 32, wherein the first end effector has a first configuration, and the second end effector has a second configuration,
wherein the first configuration and the second configuration are different from each other in at least one property selected from a group of properties consisting of: a size, a shape, and a material.

35. The electrosurgical electrode of claim 34, wherein the first end effector is an electrosurgical blade and the second end effector is ball tip type electrode.

36. The electrosurgical electrode of claim 32, wherein the first end effector has a first configuration, the second end effector has a second configuration,
wherein the first configuration and the second configuration are the same as each other in a size, a shape, and a material.

37. A method of operating an electrosurgical device, comprising:
providing an electrosurgical device, wherein the electrosurgical device comprises a housing extending from a proximal end to a distal end, and an electrosurgical electrode coupled to the housing such that the electrosurgical electrode extends from the distal end of the housing;
rotating a rotatable member relative to the housing, wherein the rotatable member is exposed on an exterior surface of the housing between the proximal end and the distal end of the housing; and
responsive to rotating the rotatable member relative to the housing, rotating the electrosurgical electrode relative the housing.

38. The method of claim 37, wherein the electrosurgical device comprises one or more housing conductors that are configured to conduct electrosurgical energy from a power cord to the electrosurgical electrode,
wherein the electrosurgical electrode comprises an electrical contact,
wherein the one or more housing conductors comprises a protrusion, and
wherein the method further comprises biasing the protrusion towards the electrical contact to electrically couple the one or more housing conductors to the electrosurgical electrode.

39. The method of claim 38, wherein the electrical contact comprises a plurality of detents around at least a portion of a circumference of the electrosurgical electrode, and
wherein the method further comprises providing, by an engagement between the protrusion and the plurality of detents, a force to the electrosurgical electrode that opposes a rotational force applied to the rotatable member.

40. The method of claim 37, wherein rotating the rotatable member comprises bi-directionally rotating the rotatable member relative to the housing to rotate the electrosurgical electrode in a first direction and a second direction, wherein the first direction is opposite the second direction.

41. The method of claim 37, wherein rotating the rotatable member comprises rotating the rotatable member in a first direction relative to the housing while preventing rotation of the rotatable member in a second direction relative to the housing, wherein the first direction is opposite the second direction.

42. The method of claim 37, wherein the electrosurgical electrode has a longitudinal axis extending between a first end and a second end, and
wherein the electrosurgical electrode has a first end effector at the first end and a second end effector at the second end.

43. The method of claim 42, further comprising coupling the electrosurgical electrode to the housing in a first state in which the first end effector in an interior cavity of the housing and the second end effector extends from the distal end of the housing.

44. The method of claim 43, further comprising, after coupling the electrosurgical electrode to the housing in the first state, (i) decoupling the electrosurgical electrode from the housing and (ii) coupling the electrosurgical electrode to the housing in a second state in which the second end effector is in the interior cavity of the housing and the first end effector extends from the distal end of the housing.

45. The method of claim 44, wherein the housing comprises an insulator defining an insulator cavity, and a receptacle that is coupled to a mount of the electrosurgical electrode,
wherein, in the first state, the first end effector is received in the insulator cavity,
wherein, in the second state, the second end effector is received in the insulator cavity,
wherein the insulator couples the rotatable member and the receptacle such that the rotatable member is rotationally fixed relative to the receptacle, and
wherein rotating the rotatable member relative to the housing comprises rotating the insulator, the receptacle, and the electrosurgical electrode coupled to the receptacle.

46. A method of forming an electrosurgical device, comprising:
forming a housing, wherein the housing extends from a proximal end to a distal end;
coupling an electrosurgical electrode to the housing such that the electrosurgical electrode extends from the distal end of the housing; and
coupling a rotatable member to the housing such that the rotatable member is exposed on an exterior surface of the housing between the proximal end and the distal end of the housing,
wherein the rotatable member is rotatable about a longitudinal axis of the housing to rotate the electrosurgical electrode relative the housing.

47. A method of operating an electrosurgical device, comprising:
providing a housing, wherein the housing extends from a proximal end to a distal end;
coupling an electrosurgical electrode to the housing such that the electrosurgical electrode extending from the distal end of the housing,
wherein the electrosurgical electrode extends between a first end and a second end, and
wherein the electrosurgical electrode has a first end effector at the first end and a second end effector at the second end.

48. The method of claim 47, wherein coupling the electrosurgical electrode to the housing comprises coupling the electrosurgical electrode to the housing in a first state in which the first end effector in an interior cavity of the housing and the second end effector extends from the distal end of the housing.

49. The method of claim 48, further comprising, after coupling the electrosurgical electrode to the housing in the first state, (i) decoupling the electrosurgical electrode from the housing and (ii) coupling the electrosurgical electrode to the housing in a second state in which the second end effector is in the interior cavity of the housing and the first end effector extends from the distal end of the housing.

50. The method of claim 49, wherein the housing comprises an insulator defining an insulator cavity, and
wherein, in the first state, the first end effector is received in the insulator cavity, and
wherein, in the second state, the second end effector is received in the insulator cavity.

51. A method of forming an electrosurgical device, comprising:
forming a housing, wherein the housing extends from a proximal end to a distal end;
forming an electrosurgical electrode, wherein the electrosurgical electrode extends between a first end and a second end, wherein the electrosurgical electrode has a first end effector at the first end and a second end effector at the second end; and
coupling the electrosurgical electrode to the housing such that the electrosurgical electrode extends from the distal end of the housing.
